(19) United States
(12) Patent Application Publication
(10) Pub. No.: US 2024/0322654 A1
(43) Pub. Date: Sep. 26, 2024

(54) Powered Surgical Tool Including A Device Housing To Facilitate Ergonomic Handling

(71) Applicant: Stryker European Operations Limited, Carrigtwohill, Co Cork (IE)

(72) Inventors: Thomas Stritch, Newmarket, Co. Cork (IE); Dustin James Payne, Kalamazoo, MI (US); Conor Mac An Tuile, Muine Bheag (IE); Daniela Burin, Cork (IE); Cian Martin, Midleton (IE); Matteo Nicolasi, Co. Cork (IE)

(73) Assignee: Stryker European Operations Limited, Carrigtwohill, Co Cork (IE)

(21) Appl. No.: 18/573,295

(22) PCT Filed: Aug. 15, 2022
(86) PCT No.: PCT/IB2022/057637

§ 371 Date: Dec. 21, 2023

Related U.S. Application Data
(60) Provisional application No. 63/367,048, filed on Jun. 26, 2022.
(60) Provisional application No. 63/232,820, filed on Aug. 13, 2021.

(51) Int. Cl. H02K 11/215; A61B 17/14; A61B 17/16; H02J 7/00

(52) U.S. Cl. CPC H02K 11/215; A61B 17/14; A61B 17/1628; H02J 7/0063

(57) ABSTRACT

A powered surgical tool (20) including a handpiece (24) including a motor and a battery and control module (21). The battery and control module includes a module housing (22) having a recess (34) for removably receiving the handpiece, the module housing defining a void space. A rechargeable battery module is disposed in the void space, the rechargeable battery module having a plurality of faces. A first printed circuit board (104) and a second printed circuit board (108) are disposed in the void space. The second printed circuit board is coupled to the first printed circuit board and arranged in a stacked configuration. The first printed circuit board and the second printed circuit board are both positioned on a same side of the rechargeable battery module. A controller is mounted to one of the printed circuit boards and configured to regulate power drawn from the rechargeable battery module based on user input.

### BACKGROUND

Handheld powered surgical tools are ubiquitous in the modern surgical theatre. Exemplary powered surgical tools include burs, drills, saws, and shavers. The devices typically include a motor and drivetrain that are sized sufficiently to meet the demands of the surgical procedure, for example, resecting cortical bone and other hardened anatomical structures. As such, a device housing needs to be correspondingly sized to accommodate the motor, the drivetrain, and other subcomponents of the device. However, it is important that the device be comfortable, ergonomic, and intuitive to the surgeon, and provide appropriate line of sight to the surgical site at which a cutting accessory assembly of the device is to be deployed. The device housing being unduly oversized or poorly weighted fails to meet such considerations.

The advent of cordless handheld powered surgical tools exacerbated the aforementioned considerations. More particularly, the device housing further needs to accommodate one or more battery cells, which may be sizable and quite heavy owing to the power and runtime required to be supplied to the motor to meet the speed and/or torque demands of the surgical procedure.

Therefore, there is a need in the art for a handheld powered surgical tool including that which overcomes one or more of the aforementioned shortcomings. There is a further need in the art for such a device that is cordless in which one or more battery cells are efficiently arranged to preserve comfort and ergonomics of the device housing.

### SUMMARY

In a feature, a powered surgical tool is described. The powered surgical tool includes a handpiece and a battery and control module. The handpiece includes a motor. The battery and control module includes a module housing having a recess for removably receiving the handpiece and defining a void space, a rechargeable battery module disposed in the void space, a first printed circuit board disposed in the void space, a second printed circuit board disposed in the void space, and a controller. The second printed circuit board is coupled to the first printed circuit board, with the second printed circuit board and the first printed circuit board being arranged in a stacked configuration. The first printed circuit board and the second printed circuit board are both positioned on a same side of the rechargeable battery module. The controller is configured to regulate power drawn from the rechargeable battery module based on user input and is mounted to at least one of the first printed circuit board and the second printed circuit board.

In another feature, a powered surgical tool is described. The powered surgical tool includes a battery and control module and a handpiece. The battery and control module includes a module housing, a rechargeable battery module, a printed circuit board, a handswitch, and a controller. The module housing has a recess for receiving the handpiece and defines a void space. The recess defines a longitudinal axis. A rechargeable battery module is disposed in the void space and includes at least one battery defining a battery longitudinal axis, the battery longitudinal axis intersecting the longitudinal axis of the recess. The printed circuit board is arranged adjacent to the rechargeable battery module. The handswitch is movably mounted to the module housing. The handswitch being configured to receive an input from a user to cause power to be drawn from the at least one battery and supplied to the motor. The controller is mounted to the printed circuit board and configured to regulate power drawn from the at least one battery and supplied to the motor based on the input from the user.

In another feature, a pencil-style powered surgical tool is described. The pencil-style powered surgical tool includes a handpiece, a handswitch, and a battery and control module. The handpiece includes a motor and defining a housing and including a tool coupler. The handpiece includes an insertion region and a grip region, the grip region defining a proximal face, and the grip region being distal the insertion region, the grip region having a larger cross-sectional area than the insertion region. The handswitch extends towards the tool coupler of the handpiece, with the handswitch reconfigurable between a first length and a second length. The battery and control module includes a module housing, a rechargeable battery module, a printed circuit board, and a controller. The module housing defines a recess for removably receiving the handpiece, the module housing including a distal face, wherein the distal face of the module housing abuts the proximal face of the grip region. The rechargeable battery module is disposed in the module housing. The printed circuit board is coupled to an inner surface of the module housing. The controller is mounted to the printed circuit board and configured to regulate power drawn from the rechargeable battery module.

In another feature, a powered surgical tool is described. The powered surgical tool includes a handpiece, a lever, and a battery and control module. The handpiece includes a motor. The lever includes magnet configured to receive an input from a user. The battery and control module includes a module housing, a rechargeable battery module, a first printed circuit board, a second printed circuit board, a handswitch sensor, and a controller. The module housing has a recess for removably receiving the handpiece. The module housing defining a void space. The rechargeable battery module is disposed in the void space; The first printed circuit board and the second printed circuit board are also disposed in the void space. The second printed circuit board is coupled to the first printed circuit board. The second printed circuit board and the first printed circuit board are arranged in a stacked configuration and are both positioned on a same side of the rechargeable battery module. The handswitch sensor is mounted to one of the first printed circuit board and the second printed circuit board and configured to output a handswitch sensor signal based on a position of the magnet of the lever. The controller is configured to regulate power drawn from the rechargeable battery module based on user input and is mounted to at least one of the first printed circuit board and the second printed circuit board.

In a feature, a powered surgical tool is described. The powered surgical tool includes a handpiece including a motor, a lever including magnet configured to receive an input from a user, and a battery and control module. The battery and control module includes a module housing, a rechargeable battery module, a first printed circuit board, a second printed circuit board, a Hall effect sensor, and a controller. The module housing has a recess for removably receiving the handpiece, the module housing defining a void space. The rechargeable battery module is disposed in the void space. The first printed circuit board is disposed in the void space. The second printed circuit board is disposed in the void space. The second printed circuit board is coupled to the first printed circuit board. The second printed circuit board and the first printed circuit board are arranged in a stacked configuration. The first printed circuit board and the second printed circuit board are both positioned on a same side of the rechargeable battery module. The Hall effect sensor is mounted to one of the first printed circuit board and the second printed circuit board and configured to output a motor sensor signal representative of a state of the motor. The controller is configured to regulate power drawn from the rechargeable battery module based on user input, the controller being mounted to at least one of the first printed circuit board and the second printed circuit board.

In a feature, a powered surgical tool comprising a handpiece including a motor, a lever, and a battery and control module. The lever including magnet configured to receive an input from a user. The battery and control module including: a module housing having a recess for removably receiving the handpiece, the module housing defining a void space; a rechargeable battery module disposed in the void space; a first printed circuit board disposed in the void space; a second printed circuit board disposed in the void space, a hall effect sensor, and a controller. The second printed circuit board is coupled to the first printed circuit board, the second printed circuit board and the first printed circuit board being arranged in a stacked configuration. The first printed circuit board and the second printed circuit board are both positioned on a same side of the rechargeable battery module. The Hall effect sensor is mounted to one of the first printed circuit board and the second printed circuit board and configured to output a motor sensor signal representative of a state of the motor. The controller is configured to regulate power drawn from the rechargeable battery module based on input from the user, the controller being mounted to at least one of the first printed circuit board and the second printed circuit board.

In a feature, a powered surgical tool including a light guide in a hermetically-sealed housing configured for sterilization is described. The powered surgical tool comprising a handpiece including a motor and a battery and control module. The battery and control module includes a module housing having a recess for removably receiving the handpiece, the module housing defining a void space, the module housing including a light guide, the light guide defines a base and a plurality of protrusions extending from the base, and the base of the light guide defines an aperture between the protrusions, the light guide being transparent, a surface of the protrusions defining a portion of an outermost surface of the battery and control module. The battery and control module also includes a filler polymer disposed in the aperture, the filler polymer being opaque. The battery and control module also includes a rechargeable battery module disposed in the void space. The battery and control module also includes a printed circuit board disposed in the void space. The battery and control module also includes a plurality of light emitting diodes coupled to the printed circuit board, each of the plurality of light emitting diodes aligned with a respective one of the plurality of protrusions so that light emitted from a light emitting diode does not interfere with light emitted from an adjacent light emitting diode. The battery and control module includes a controller disposed in the void space and configured to regulate power drawn from the rechargeable battery module, the controller being mounted to the printed circuit board.

A method of forming a light guide in a housing of a shell component of a powered surgical tool, the shell component including a hermetically-sealed housing configured for sterilization, the method including providing a plurality of light emitting diodes on a circuit board mounted in a first portion of a shell component. The method also includes providing a light guide including a plurality of protrusions and defining an aperture between the plurality of protrusions, the light guide being transparent. The method also includes positioning light guide such that the protrusions are aligned with of LEDS and such that light guide contacts the circuit board. The method also includes applying a molten filler material such that the molten filler material fills the aperture and contacts each of the protrusions, wherein the molten filler material is opaque.

A powered surgical tool having a pistol grip is described. The powered surgical tool includes a handpiece including a motor and a battery and control module. The battery and control module includes a module housing defining a barrel portion and a handle portion, the barrel portion having a recess for removably receiving the handpiece, the handle portion extending downwardly from the barrel portion, the module housing defining a void space. The battery and control module also includes a rechargeable battery module disposed in the void space. The battery and control module also includes a first printed circuit board disposed in the void space. The battery and control module also includes a second printed circuit board disposed in the void space, the second printed circuit board being coupled to the first printed circuit board, the second printed circuit board and the first printed circuit board being arranged in a stacked configuration. The first printed circuit board and the second printed circuit board are both positioned on a same side of the rechargeable battery module. The battery and control module also includes a Hall-effect sensor disposed in the void space and being mounted to one of the first and the second printed circuit board and configured to output a motor sensor signal representative of a state of the motor. The battery and control module also includes a controller disposed in the void space, the controller being configured to regulate power drawn from the rechargeable battery module based on user input, the controller being mounted to at least one of the first printed circuit board and the second printed circuit board.

In a feature, a compact battery powered surgical tool for driving orthopedic tools while being sterilizable is described. The tool includes a handpiece including a motor and a battery and control module, the battery and control module hermetically sealed for sterilization. The battery and control module includes a module housing having a recess for receiving the handpiece, the module housing defining a void space and including a vent assembly for venting of the void space during sterilization. The battery and control module also includes a rechargeable battery module disposed in the void space, the rechargeable battery module including at least three battery cells, each of the battery cells including an onset of thermal runaway temperature rating ranging between 150 and 180 degrees Celsius. The battery and control module also includes a printed circuit board arranged adjacent to the rechargeable battery module. The battery and control module also includes a handswitch coupled to one of the battery and control module and the handpiece, the handswitch being configured to receive an input from a user to cause power to be drawn from the at least three battery cells and supplied to the motor. The battery and control module also includes a controller configured to regulate power drawn from the at least three battery cells and supplied to the motor based on the input from the user, wherein the controller is mounted to the printed circuit board. the powered surgical tool exhibits a mechanical output power of at least 90 Watts while the battery and control module has a volume of less than 325 cm³.

In a feature, a powered surgical tool is described. The powered surgical tool includes a handpiece including a housing and a motor disposed within the housing, the housing including a first protrusion including a first tapered surface (a) and a second protrusion (c) include a second tapered surface. The powered surgical tool also includes a battery and control module. The battery and control module includes a module housing having a surface that defines a recess for removably receiving the handpiece, the module housing defining a void space, the surface includes a first channel (b) that includes a third tapered surface that is complementary in shape to the first tapered surface of the first protrusion. The battery and control module also includes a rechargeable battery module disposed in the void space, a printed circuit board disposed in the void space, a controller disposed in the void space and configured to regulate power drawn from the rechargeable battery module, the controller being mounted the printed circuit board. The battery and control module also includes a latch assembly including a lock member and a biasing member, the biasing member positioned to urge the lock member towards the recess, and the lock member defining a fourth tapered surface. The powered surgical tool is configured such that when the handpiece is fully seated within the recess and the latch assembly is in an engaged state, the lock member is biased by the biasing member such that the fourth tapered surface is actively urged against the second tapered surface and the first tapered surface is actively urged against the third tapered surface such that vibration and noise is reduced from an interaction between the handpiece and the battery and control module.

In a feature, a method of forming a hermetic seal with conductive terminals in a control module of a powered surgical tool is described. The control module being hermetically-sealed and configured for sterilization. The method includes providing at least three conductive terminals, each of the conductive terminals having a first end and a second end and a central region between the first end and the second end. The method also includes mounting the first end of the at least three conductive terminals in a mold fixture such at the second end of the at least three conductive terminals extend outward from the mold fixture, the at least three conductive terminals are positioned in the mold fixture to define an array of conductive terminals surrounding a central region between the at least three conductive terminals. The method also includes injecting a molten filler material in the central region of the array of conductive terminals while the conductive terminals are mounted in the mold fixture such that the molten filler material surrounds the central region of each of the at least three conductive terminals and form a hermetic seal therewith.

In a feature, a control module for regulating operation of an electric motor of a surgical device, is described. The control module includes a housing, a circuit board disposed in the housing for regulating operation of an electric motor; and at least three conductive terminals that extends through the housing for establishing an electrical connection between the circuit board and the electric motor, wherein the housing directly contacts at least three conductive terminals to form a housing-terminal interface, and the housing is formed from a thermoplastic material.

Further areas of applicability of the present disclosure will become apparent from the detailed description, the claims, and the drawings. The detailed description and specific examples are intended for purposes of illustration only and are not intended to limit the scope of the disclosure.

### BRIEF DESCRIPTION OF THE DRAWINGS

Advantages of the present disclosure will be readily appreciated as the same becomes better understood by reference to the following detailed description when considered in connection with the accompanying drawings.

FIG. 1 is a perspective view of a powered surgical tool according to four implementations of the present disclosure.

FIG. 2 is a perspective view of a first implementation of the powered surgical tool shown with a first cutting accessory assembly attached, according to the teachings of the present disclosure.

FIG. 3 is a perspective view of a first implementation of the powered surgical tool shown with a second cutting accessory assembly attached, according to the teachings of the present disclosure.

FIG. 4 is an elevation view of a first implementation of the powered surgical tool of FIG. 2, according to the teachings of the present disclosure.

FIG. 5 is a perspective view of a first implementation of the powered surgical tool of FIG. 2, according to the teachings of the present disclosure.

FIG. 6 is a perspective view of a first implementation of the powered surgical tool of FIG. 2, according to the teachings of the present disclosure.

FIG. 7 is a perspective view of a first implementation of the powered surgical tool of FIG. 2, according to the teachings of the present disclosure.

FIG. 8 is an exploded view of a first implementation of the powered surgical tool of FIG. 2, according to the teachings of the present disclosure.

FIG. 9 depicts a perspective view of a handswitch assembly of a first implementation of the powered surgical tool of FIG. 2, according to the teachings of the present disclosure.

FIG. 10 depicts a perspective view of a handswitch assembly in an extended position of a first implementation of the powered surgical tool of FIG. 2, according to the teachings of the present disclosure.

FIG. 11 depicts a perspective view of a handswitch assembly of a first implementation of the powered surgical tool of FIG. 2, according to the teachings of the present disclosure.

FIG. 12 depicts an elevation view of a handswitch assembly of a first implementation of the powered surgical tool of FIG. 2, according to the teachings of the present disclosure.

FIG. 13 depicts a perspective view of a first implementation of a portion of the powered surgical tool of FIG. 2 and a latch assembly, according to the teachings of the present disclosure.

FIG. 14 depicts a perspective view of a first implementation of the powered surgical tool of FIG. 2 and a latch assembly, according to the teachings of the present disclosure.

FIG. 15 depicts an elevation view of a first implementation of the powered surgical tool of FIG. 2 and a latch assembly, according to the teachings of the present disclosure.

FIG. 16 depicts a cross sectional view of a first implementation of the powered surgical tool of FIG. 2, according to the teachings of the present disclosure.

FIG. 17 depicts a perspective view of a first printed circuit board and a second printed circuit board of a first implementation of the powered surgical tool of FIG. 2, according to the teachings of the present disclosure.

FIG. 18 depicts a bottom view of a first printed circuit board and a second printed circuit board of a first implementation of the powered surgical tool of FIG. 2, according to the teachings of the present disclosure.

FIG. 19 depicts an elevation view of a first printed circuit board and a second printed circuit board of a first implementation of the powered surgical tool of FIG. 2, according to the teachings of the present disclosure.

FIG. 20 depicts a top view of a first printed circuit board and a second printed circuit board of a first implementation of the powered surgical tool of FIG. 2, according to the teachings of the present disclosure.

FIG. 21 depicts a cross section view of a flared portion of a first implementation of the powered surgical tool of FIG. 2, according to the teachings of the present disclosure.

FIG. 22 depicts a cross section view of a base portion of a first implementation of the powered surgical tool of FIG. 2, according to the teachings of the present disclosure.

FIG. 23 depicts a cross section view of a flared portion with components removed of a first implementation of the powered surgical tool of FIG. 2, according to the teachings of the present disclosure.

FIG. 24 depicts a cross section view of a handpiece inserted into a recess of a flared portion of a first implementation of the powered surgical tool of FIG. 2, according to the teachings of the present disclosure.

FIG. 25 depicts a perspective view of a first handpiece with a cutting accessory of a first implementation of the powered surgical tool of FIG. 2, according to the teachings of the present disclosure.

FIG. 26 depicts a perspective view of a first implementation of a main casing of the handpiece of FIG. 25, according to the teachings of the present disclosure.

FIG. 27 depicts a cross sectional view of a first handpiece of FIG. 25 of a first implementation of the powered surgical tool of FIG. 2, according to the teachings of the present disclosure.

FIG. 28 depicts a perspective view of a second implementation of a main casing of the handpiece of FIG. 25, according to the teachings of the present disclosure.

FIG. 29 depicts a perspective view of a third implementation of a main casing of the handpiece of FIG. 25, according to the teachings of the present disclosure.

FIG. 30 is an elevation view of a second implementation of the powered surgical tool, according to the teachings of the present disclosure.

FIG. 31 is an exploded perspective view of the second implementation of the powered surgical tool of FIG. 30, according to the teachings of the present disclosure.

FIG. 32 is a sectional elevation view of a battery and control module of the powered surgical tool of FIG. 30, according to the teachings of the present disclosure.

FIG. 33 is an elevation view of a third implementation of the powered surgical tool, according to the teachings of the present disclosure.

FIG. 34 is an exploded perspective view of the powered surgical tool of FIG. 33, according to the teachings of the present disclosure.

FIG. 35 is a sectional elevation view of the battery and control module of the powered surgical tool of FIG. 33, according to the teachings of the present disclosure.

FIG. 36 is an elevation view of a fourth implementation of the powered surgical tool, according to the teachings of the present disclosure.

FIG. 37 is an exploded perspective view of the powered surgical tool of FIG. 3, according to the teachings of the present disclosure.

FIG. 38 is a sectional elevation view of the battery and control module of the powered surgical tool of FIG. 36, according to the teachings of the present disclosure.

FIG. 39 is a perspective view of fifth implementation of the powered surgical tool in which a device housing of the battery and control module of FIG. 30 is modified to be shorter in length, according to the teachings of the present disclosure.

FIG. 40 is a perspective view of sixth implementation of the powered surgical tool in which the device housing of the battery and control module of FIG. 32 is modified to be shorter in length, according to the teachings of the present disclosure.

FIG. 41 is a perspective view of seventh implementation of the powered surgical tool in which an upper aspect of a proximal portion of the battery and control module of FIG. 36 is modified, according to the teachings of the present disclosure.

FIG. 42 is a perspective view of an eighth implementation of the powered surgical tool in which the device housing is arranged in a pistol grip configuration, according to the teachings of the present disclosure.

FIG. 43 is a perspective view of an eighth implementation of the powered surgical tool in which the device housing is arranged in a pistol grip configuration, according to the teachings of the present disclosure.

FIG. 44 is cross-sectional view of an eighth implementation of the powered surgical tool shown in FIG. 43 in which the device housing is arranged in a pistol grip configuration, according to the teachings of the present disclosure.

FIG. 45 is a perspective view of an eighth implementation of the powered surgical tool shown in FIG. 43 in which the device housing is arranged in a pistol grip configuration, according to the teachings of the present disclosure.

FIG. 46 is a perspective view of an eighth implementation of the powered surgical tool in which the device housing is arranged in a pistol grip configuration, according to the teachings of the present disclosure.

FIG. 47 is a sectional plan view of the powered surgical tool of FIG. 46 taken along lines 47-47, according to the teachings of the present disclosure.

FIG. 48 is a perspective view of the eight implementation of the battery and control module of the powered surgical tool in which the device housing is arranged in the pistol grip configuration, according to the teachings of the present disclosure.

FIG. 49 is a sectional elevation view of the powered surgical tool of FIG. 48 taken along lines 49-49, according to the teachings of the present disclosure.

FIG. 50 is a rear view of a flared portion of the battery and control module of a first implementation of the powered surgical tool of FIG. 2, according to the teachings of the present disclosure.

FIG. 51 is a cross sectional view of a first implementation of an interface between conductive terminals and a device housing of a battery and control module of a first implementation of the powered surgical tool of FIG. 2, according to the teachings of the present disclosure.

FIG. 52 is a cross sectional view of an alternative configuration of an interface between conductive terminals and the device housing of a battery and control module of a first implementation of the powered surgical tool of FIG. 2, according to the teachings of the present disclosure.

FIG. 53 is a view of a light guide of a battery and control module of a first implementation of the powered surgical tool of FIG. 2, according to the teachings of the present disclosure.

FIG. 54 is a view of a light guide of a battery and control module of a first implementation of the powered surgical tool of FIG. 2, according to the teachings of the present disclosure.

FIG. 55 is a view of an interface between a plurality of ribs of a light guide and a plurality of light emitting diodes of a battery and control module of a first implementation of the powered surgical tool of FIG. 2, according to the teachings of the present disclosure.

FIG. 56 is a cross sectional view of an interface between a printed circuit board and one of the plurality of ribs of a light guide of a battery and control module of a first implementation of the powered surgical tool of FIG. 2, according to the teachings of the present disclosure.

FIG. 57 is a cross sectional view of a light guide and a plurality of light emitting diodes of a battery and control module of a first implementation of the powered surgical tool of FIG. 2, according to the teachings of the present disclosure.

FIG. 58 is a view of a light guide of a battery and control module of a first implementation of the powered surgical tool of FIG. 2, according to the teachings of the present disclosure.

FIG. 59 is an exploded view of a first implementation of the powered surgical tool of FIG. 2 depicting a latch assembly and features of the handpiece and features of the battery and control module that cooperate with the latch assembly, according to the teachings of the present disclosure.

FIG. 60 is a cross sectional elevation view of a first implementation of the powered surgical tool of FIG. 2 depicting a latch assembly and features of the handpiece and features of the battery and control module that cooperate with the latch assembly, according to the teachings of the present disclosure.

FIG. 61 is a view of a first implementation of the powered surgical tool of FIG. 2 depicting a latch assembly and features of the handpiece and features of the battery and control module that cooperate with the latch assembly, according to the teachings of the present disclosure.

FIG. 62 is a cross sectional plan view of a first implementation of the powered surgical tool of FIG. 61 depicting a latch assembly and features of the handpiece and features of the battery and control module that cooperate with the latch assembly, the sectional plan view taken along lines 61-61, according to the teachings of the present disclosure.

FIG. 63A depicts a first configuration of a first printed circuit board, a second printed circuit board, a third printed circuit board, and a fourth printed circuit board of an eight implementation of the powered surgical tool of FIG. 42, according to the teachings of the present disclosure.

FIG. 63B depicts a second configuration of a first printed circuit board, a second printed circuit board, a third printed circuit board, and a fourth printed circuit board of an eight implementation of the powered surgical tool of FIG. 42, according to the teachings of the present disclosure.

FIG. 64a is a view of mold fixture and conductive terminals of a first implementation of the powered surgical tool of FIG. 2, according to the teachings of the present disclosure.

FIG. 64b is a plan view of mold fixture and conductive terminals of a first implementation of the powered surgical tool of FIG. 2, according to the teachings of the present disclosure.

### DETAILED DESCRIPTION

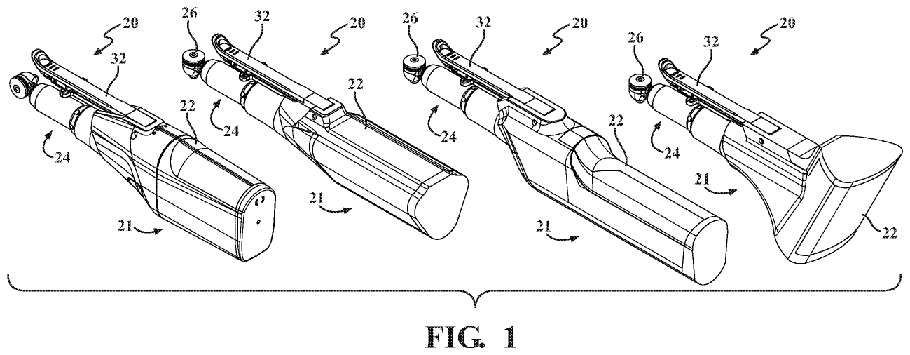

FIG. 1 shows four implementations of a powered surgical tool 20 in which a device housing 22 of a battery and control module 21 is designed to provide improved ergonomics and usability. The powered surgical tool 20 includes a handpiece 24 configured to be removably coupled with the battery and control module 21. The handpiece 24 may include a motor and a drivetrain (not identified), and also includes additional subcomponents such as electrical sockets, a gearbox, and geometries to removably receive a cutting accessory including a head 57. Other than certain features of the handpiece 24 to be described in further detail, the handpiece 24 may take a form disclosed in commonly-owned International Publication No. WO 2013/177423, published Nov. 28, 2013, the entire contents of which are hereby incorporated by reference, and sold under the tradename F1 by Stryker Instruments (Kalamazoo, Mich.).

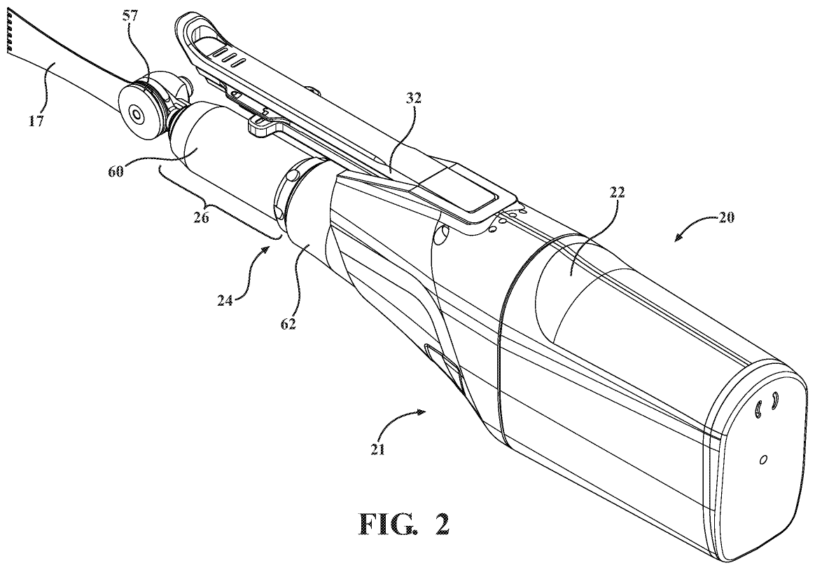

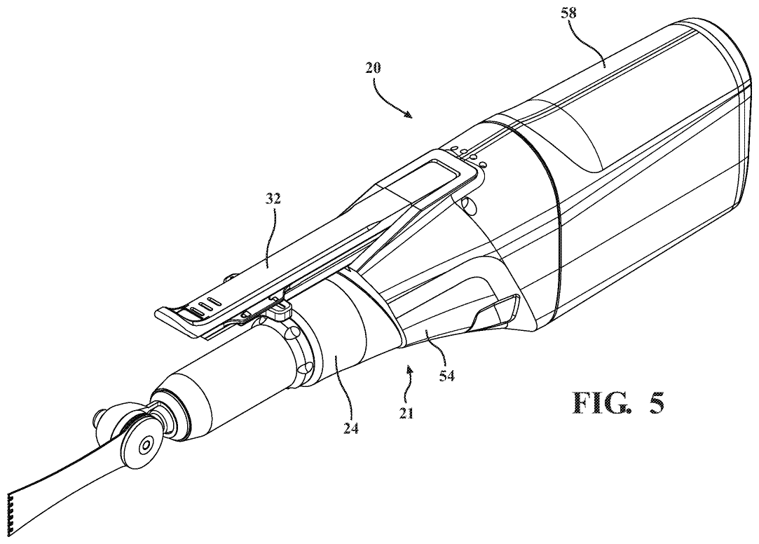

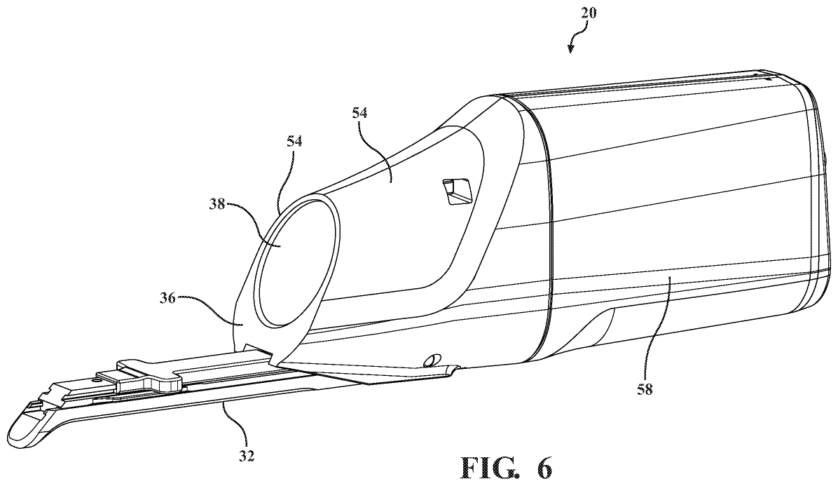

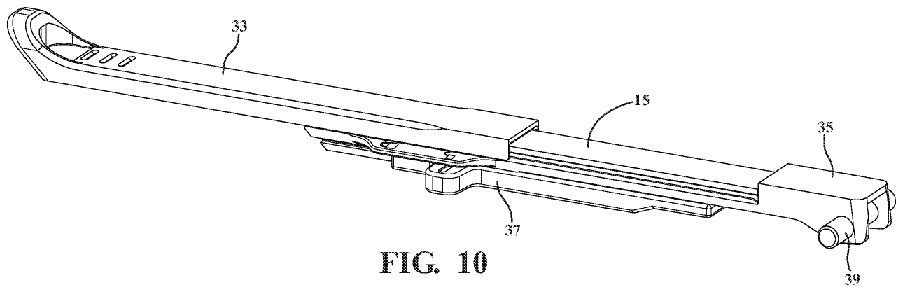

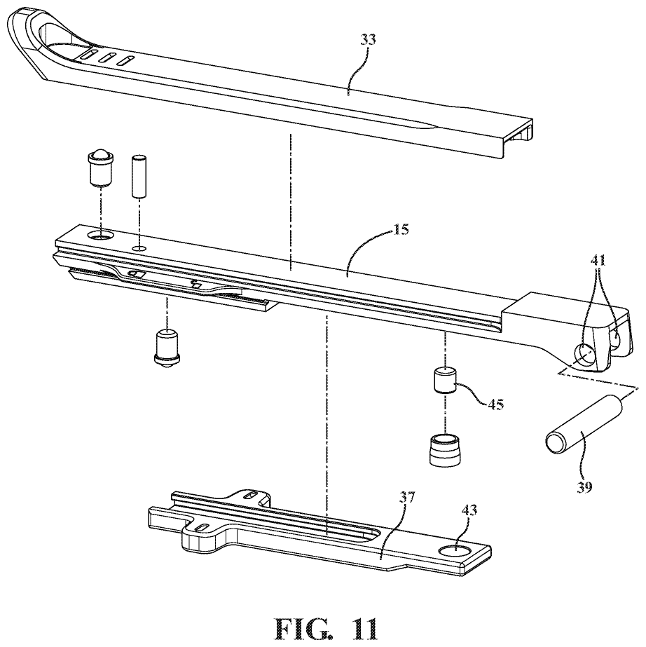

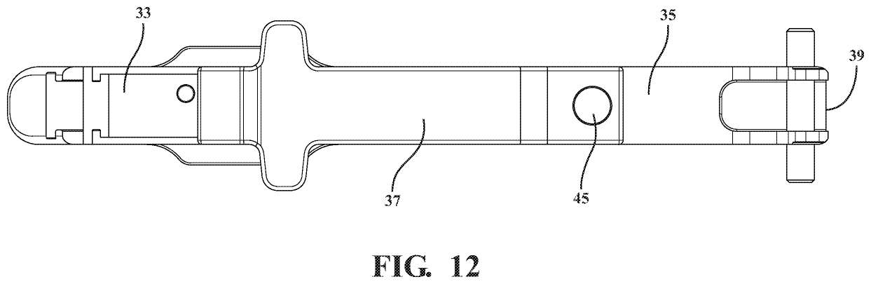

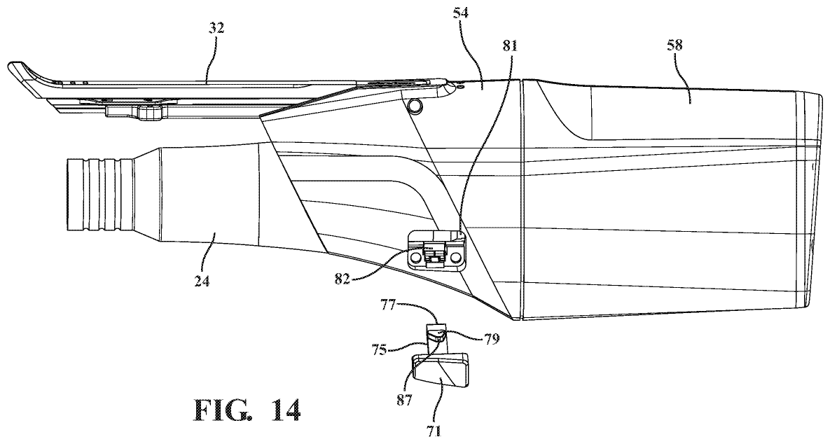

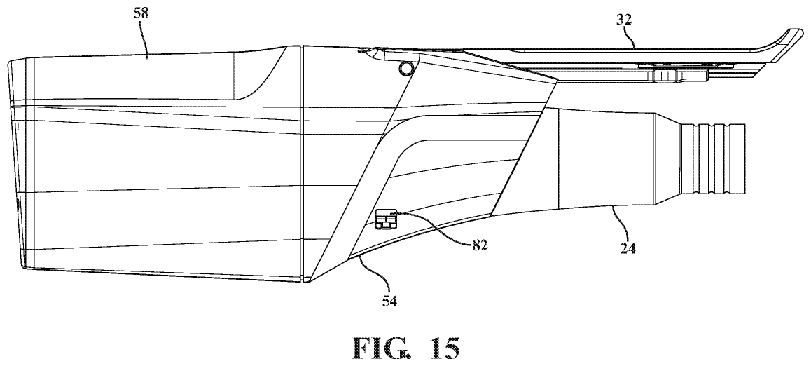

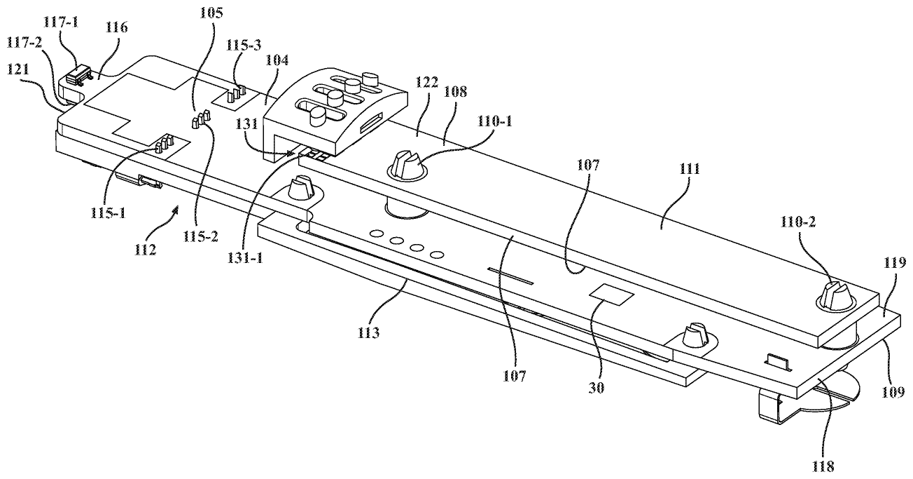

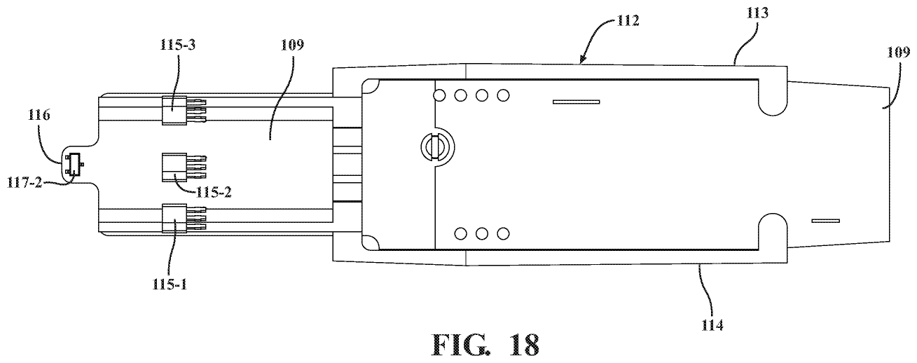

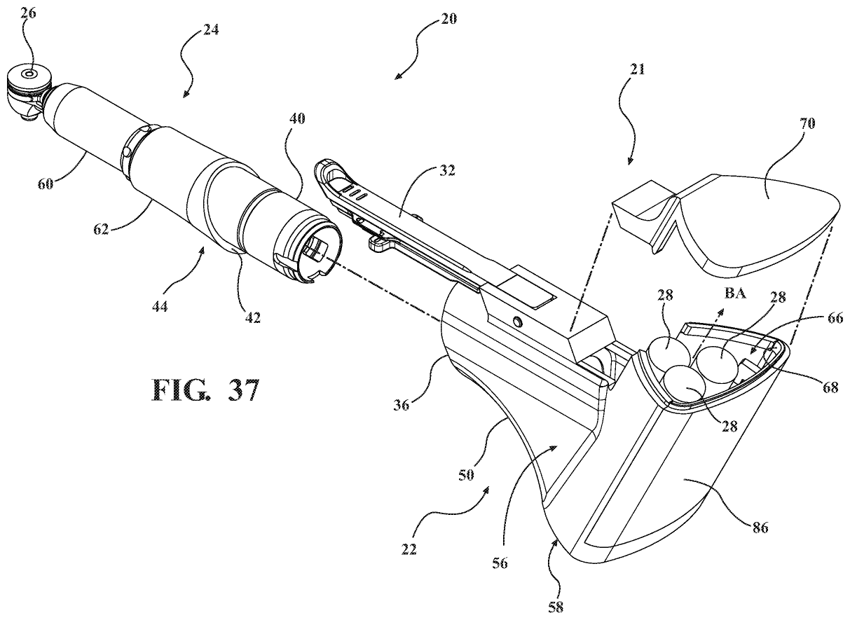

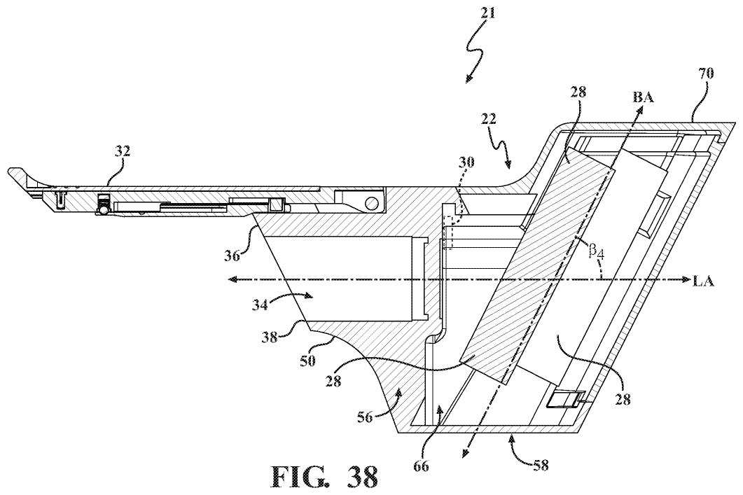

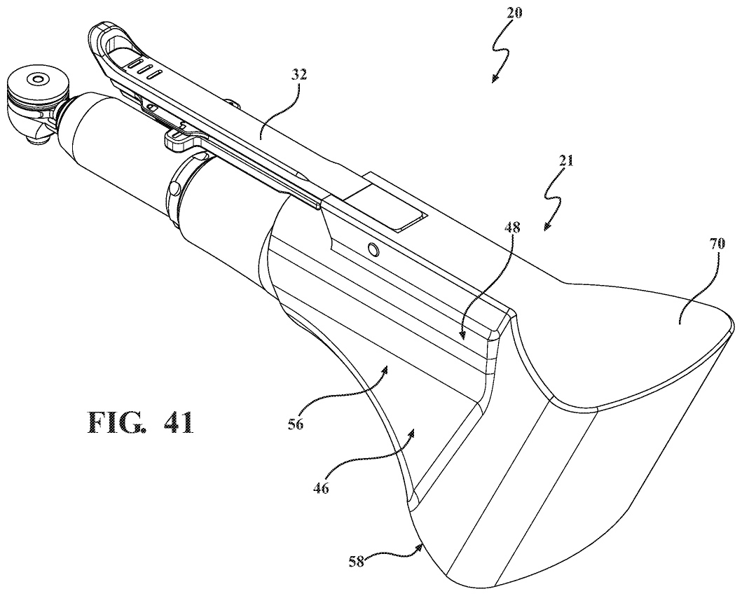

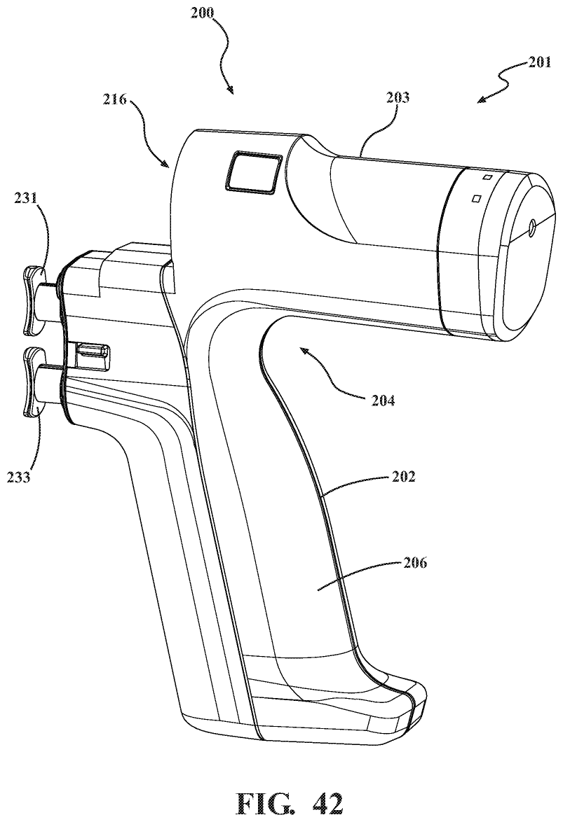

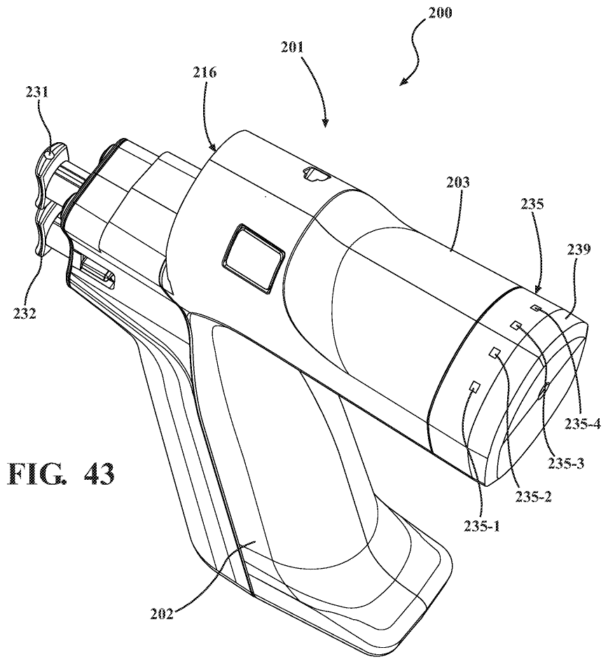

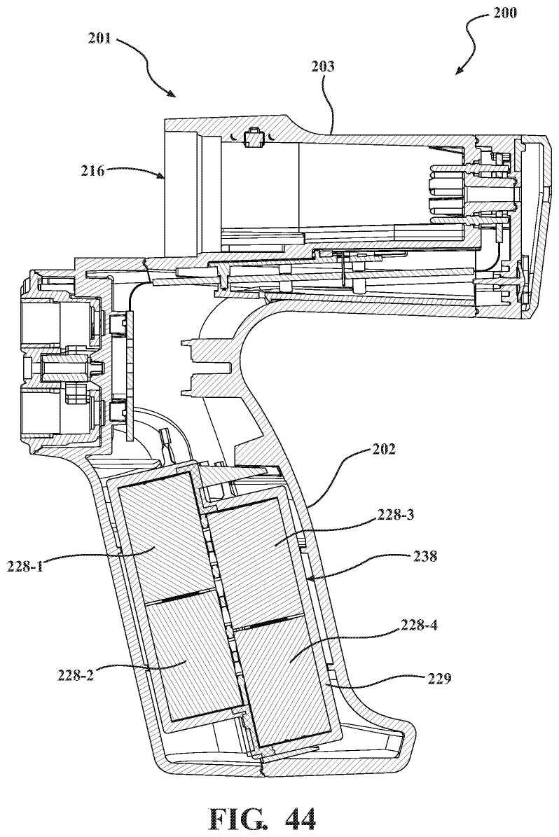

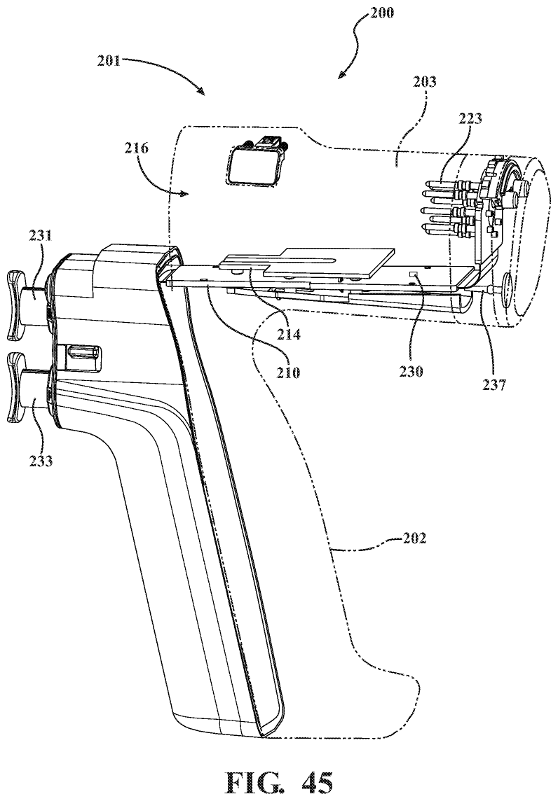

The energy applicator 17 is configured to be deployed at a surgical site to manipulate tissue with operation of the powered surgical tool 20. The cutting accessory assembly 26 may be unique to complementary versions of the motor and the drivetrain so as to provide a set of handpieces 24 configured to be selectively and interchangeably coupled with the battery and control module 21. With additional reference to FIG. 2, the powered surgical tool 20 is shown with a sagittal saw attached to the cutting accessory assembly 26. The medical professional may use the sagittal saw blade for cutting bones, for example small bones of a hand or foot of a patient, ligaments or other tissue. Any device or accessory which is applied to the surgical site, whether it be a sagittal saw blade or drill bit, may generally be referred to as an energy applicator 17 throughout. In other embodiments, the powered surgical tool 20 may be a rotary drill, reamer, wire driver, oscillating or reciprocating saw, ultrasonic device or photonic device. Likewise, the energy applicator 17 may be a drill bit, bur, saw, reamer, grinding disc, ultrasonic cutting or catheterization tip, laser, etc. The type of tool used is not intended to limit the present invention. The motor may be a universal motor which may interchangeably receive more than one cutting accessory assembly 26 as described below. It is further appreciated that the set of handpieces may be interchangeably coupled with the battery and control module 21 providing for a pencil grip configuration (FIGS. 1-41), and the battery and control module 21 providing for a pistol grip configuration (FIGS. 42-49). The powered surgical tool 20 of the present disclosure may be particularly well suited for orthopedic procedures involving the arm, hand, leg, foot, mandible, and skull, but other small bone orthopedic and soft tissues procedures are contemplated.

The medical professional may use the sagittal saw blade for cutting bones, for example small bones of a hand or foot of a patient, ligaments or other tissue. Any device or accessory which is applied to the surgical site, whether it be a sagittal saw blade or drill bit, may generally be referred to as an energy applicator 17 throughout. The cutting accessory assembly 26 as shown includes a head 57 which is moveable between an open position in which the energy applicator 17 may be removed, exchanged, or inserted and a closed position in which the energy applicator 17 is inserted into the head 57 and locked into place. The cutting accessory assembly 26 may have certain features described in U.S. Pat. No. 7,833,241, entitled, “Surgical Saw Blade Coupler,” the contents which are hereby incorporated by reference in their entirety.

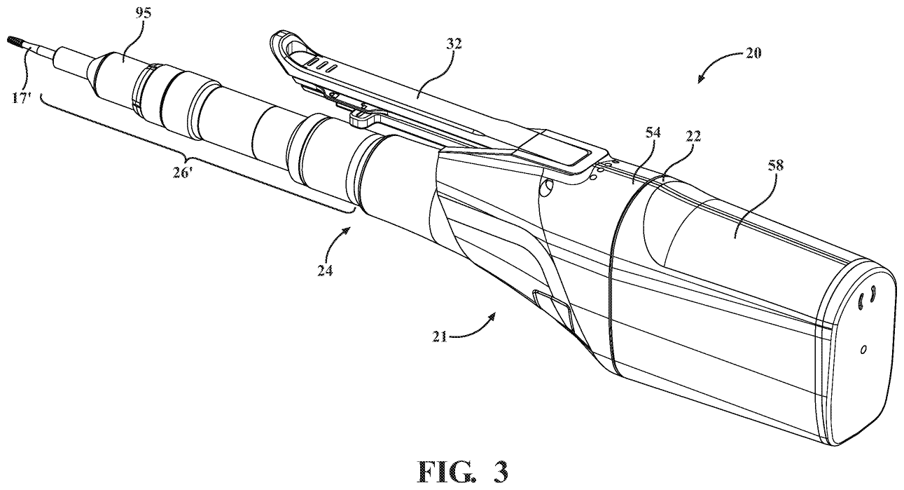

In FIG. 3, the powered surgical tool 20 is shown configured as a rotary drill. As such, the cutting accessory 26′ is shown to include a drill chuck 85 which receives the energy applicator 17′ shown as a drill bit. In other implementations, the powered surgical tool 20, may be configured as a reamer, a wire driver, ultrasonic device, or photonic device and as such the energy applicator 17 may be a bur, saw, reamer, grinding disc, ultrasonic cutting or catheterization tip, laser, etc. The type of tool used is not intended to limit the present invention.

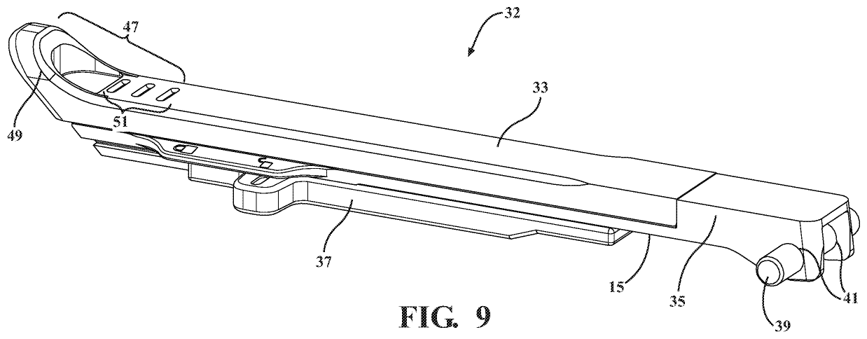

The battery and control module 21 includes at least one battery cell 28, and a motor controller 30 that is coupled to a printed circuit board as will be discussed in greater detail below. The motor controller 30 is in communication with the battery 28, a motor sensor and a handswitch sensor, and further configured to be arranged in communication with the motor when the handpiece 24 is removably coupled to the battery and control module 21. The battery and control module 21 may include a handswitch assembly 32 optionally coupled to the device housing 22 and configured to receive an input from a user to operate the powered surgical tool 20. For example, the handswitch assembly 32 may be spring-loaded and include a magnet such that, when the handswitch assembly 32 is actuated, the magnet is moved towards a handswitch sensor (discussed in greater detail with respect to FIGS. 9-12) in the device housing 22. While the example is provided that the handswitch assembly 32 is coupled to the battery and control module 21, the handswitch assembly 32 may be detachable and attached to any portion of the battery and control module 21 and/or handpiece 24. Further, the handswitch assembly 32 may be part or attached to the handpiece 24.

The motor controller 30 receives a signal from the handswitch sensor, and causes power to be drawn from at least one battery cell 28 to be supplied to the motor. The motor controller 30 is configured to determine a rotational position of the rotor of the motor, and control the motor based on the rotary position of the rotor as sensed by the motor sensors. In alternative implementations, one or more handswitch sensors may not be included and the motor may be controlled through other means. An operating speed of the powered surgical tool 20 may be increased incrementally as the handswitch assembly 32 is actuated between a default position and fully engaged position. Additional features of the motor controller 30, the handswitch assembly 32, and other electronic subcomponents of the powered surgical tool 20 may be disclosed in the aforementioned International Publication No. WO 2013/177423.

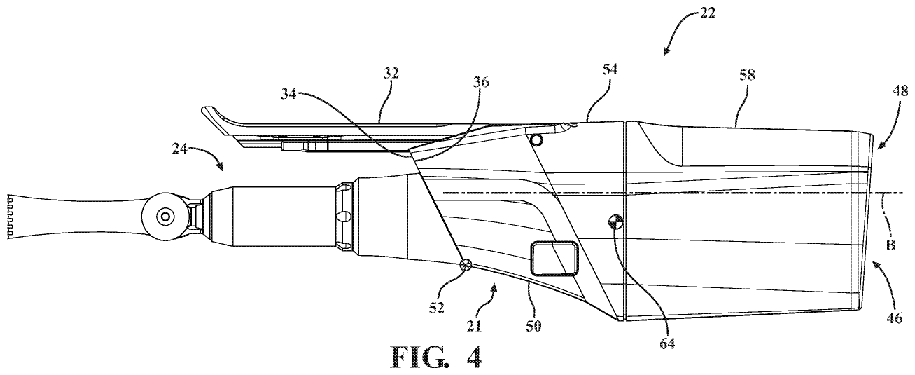

Referring now to FIGS. 2-24, the first implementation of the powered surgical tool 20 is shown. With particular reference to FIGS. 4-8 and 16, the device housing 22 defines a recess 34 sized to removably receive the handpiece 24. More particularly, the device housing 22 includes a front surface 36 defining an opening 38 that extends proximally to define the recess 34. The recess 34 may be at least substantially cylindrical to be contoured to a hub 40 of the handpiece 24. The hub 40 of the handpiece 24 is disposed within the recess 34 to establish communication between the motor of the handpiece 24 and the battery and control module 21. Several subcomponents of the device housing 22 may be disposed within or adjacent to the recess 34 to releasably secure the handpiece 24 to the device housing 22 and establish the communication between the motor and the motor controller 30. The subcomponents may include one or more terminals 23, such as contact pins 23, that provide the connection for the battery and control module 21 to the handpiece 24. Such subcomponents may also include a latch and sensors as discussed in greater detail below. Some of such subcomponents are disclosed in the aforementioned International Publication No. WO 2013/177423, the contents which have previously been incorporated by reference.

When oriented to be supported by the hand of the user in the pencil grip configuration, the device housing 22 may be considered to include a lower aspect 46 and an upper aspect 48. The lower aspect 46 may be one or more surfaces or geometries that generally define a lower portion of the device housing 22, and the upper aspect 48 may be one or more surfaces or geometries that generally define an upper portion of the device housing 22. FIG. 4 identifies a boundary (B) between the lower aspect 46 and the upper aspect 48; however, it should be understood that the identified boundary is for convention only in describing relative positioning of features of the device housing 22. The handswitch assembly 32 may be coupled to the upper aspect 48, as will be discussed in greater detail below.

The lower aspect 46 of the device housing 22 may include a surface 50 that is arcuate to define a saddle region 52 sized to accommodate a web of the hand of the user in the pencil grip configuration. With the handpiece 24 being configured to be supported by digits of the hand of the user in the pencil grip configuration, the device housing 22 cooperates with the handpiece 24 to provide for the pencil grip configuration that improves ergonomics and locates a center of mass of the powered surgical tool 20 in an optimal manner to improve handling of the powered surgical tool 20.

The surface 50 may be contoured to an adjacent portion of the handpiece 24 to define the saddle region 52. More particularly, the device housing 22 may include a flared portion 54 extending from the front surface 36, and a base portion 58 extending from the flared portion 54. The base portion 58 and the flared portion 54 may be coupled to each other. In some instances the base portion 58 and the flared portion 54 may be coupled to each other and hermetically sealed. The flared portion 54 tapers in a distal direction or flares outwardly in a proximal direction (here “proximal” is understood to mean towards the practitioner holding the powered surgical tool 20, away from the site to which the energy applicator 17 is applied. “Distal” is understood to mean away from the practitioner holding the powered surgical tool 20, towards the site to which the energy applicator 17 is applied). The distal end of the flared portion maybe referred to as a tapered end 61. The base portion 58 may have an axial section that is at least substantially constant.

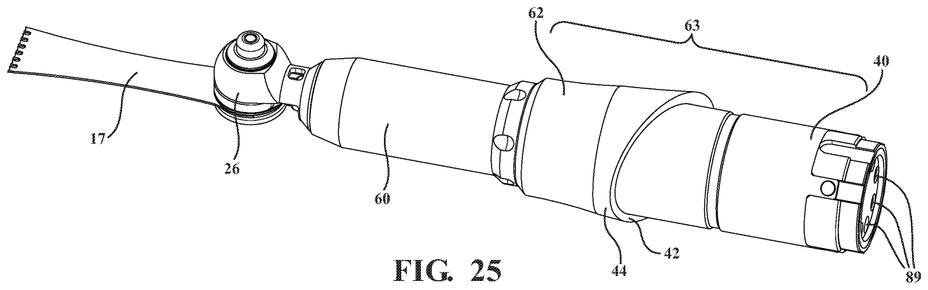

With continued reference to FIG. 4 and additional reference to FIGS. 25-29, the handpiece 24 is shown. The handpiece 24 includes a main casing 63 which is coupled to the cutting accessory assembly 26 which includes a distal barrel 60 coupled to the accessory assembly 26 that receives the energy applicator 17. The main casing 63 houses the motor and additional components as discussed in International Publication No. WO 2013/177423, published Nov. 28, 2013, the entire contents of which were previously incorporated by reference. The main casing 63 may include a proximal barrel 62, a neck 67, a sleeve 44, and a hub 40. The neck 67 is cylindrical in shape and has a diameter that is smaller than a diameter of the proximal barrel 62 or a diameter of the hub 40. The neck 67 includes one or more features which facilitate the coupling of the main casing 63 to the cutting accessory assembly 26, in particular, the distal barrel 60. The hub 40 tapers in the proximal direction. The proximal end of the hub 40 may include one or more apertures for receiving the conductive terminals 23, such as conductive pins, to connect the battery and control module 21 to the motor of the handpiece 24. The sleeve 44 may define a lip 42. When the hub 40 of the handpiece 24 is disposed within the recess 34, the lip 42 of a sleeve 44 of the handpiece 24 may be positioned adjacent to or abutting the front surface 36 of the device housing 22. Stated differently, no portion of the sleeve 44 gets inserted into the recess 34 since the lip 42 may abut the front surface 36. The lip 42 may also be spaced apart from the front surface 36 or abut the front surface 36 from the inside.

The flared portion 54 may be contoured to the sleeve 44 of the handpiece 24 with the handpiece 24 removably coupled to the device housing 22. The sleeve 44 of the handpiece 24 includes an outward taper in the proximal direction such that an interface or transition between the sleeve 44 and the flared portion 54 appears and feels smooth and uninterrupted. Stated differently a portion of a proximal end of the sleeve 44 that abuts the upper aspect 48 of the flared portion 54 is less thick than a portion of a proximal end of the sleeve 44 that abuts the lower aspect 46 of the flared portion 54. With particular reference to FIG. 27, since the sleeve 44 has an outward taper, the sleeve 44 has a cross-sectional area that is non-circular. In such an arrangement, the smooth transition of the saddle region 52 advantageously accommodates hands of different sizes as well as different options for the user to comfortably position the hand along the powered surgical tool 20. For example, the user may prefer to pinch a distal barrel 60 of the handpiece 24 and support the sleeve 44 of the handpiece 24 with the web of the hand. With reference back to FIG. 4, With the web of the hand supporting the powered surgical tool 20 at saddle region 52, for example, a center of mass 64 of the powered surgical tool 20 is moved closer to the saddle region 52. Instances where the center of mass 64 is presently identified in the figures are to be understood as approximate locations, and further understood to constitute approximate locations of the center of mass 64 with or without all aspects of the handpiece 24 removably coupled with the battery and control module 21. For the center of mass 64 for a battery and control module 21 with all aspects of the handpiece 24 coupled to the battery and control module 21 may have a center of mass 64 that is more distal than may be indicated with the battery and control module 21 when no handpiece 24 is coupled to the battery and control module 21. The weight of the powered surgical tool 20 can better be supported by the web of the hand of the user when the center of mass 64 is located more distal.

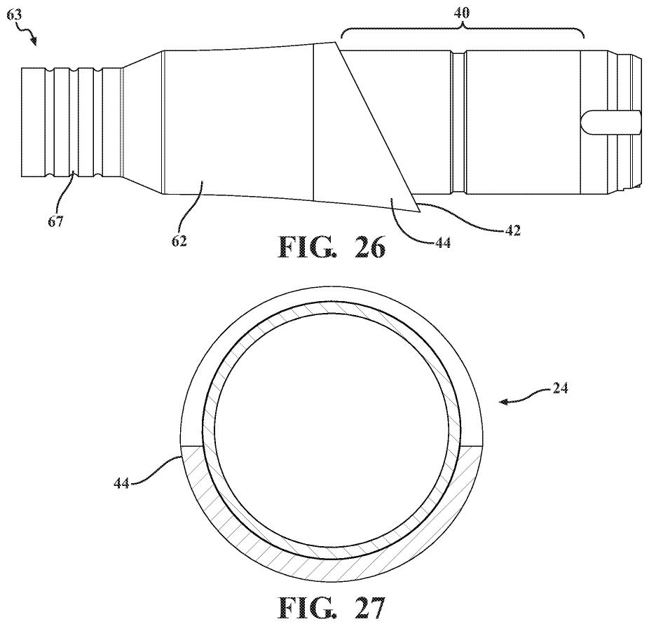

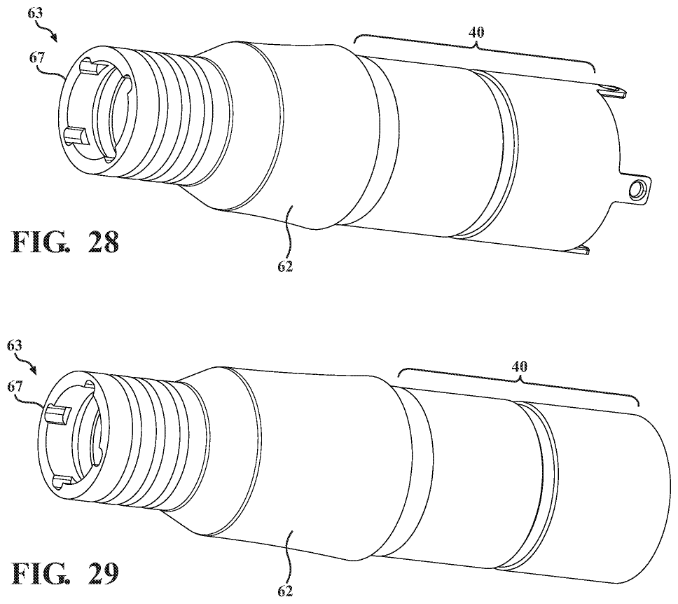

With particular reference to FIGS. 28 and 29, a second implementation of the main casing 63 of the handpiece 24 and a third implementation of the main casing 63 of the handpiece 24 are depicted. With the various implementation of the powered surgical tool 20 like features are indicated by the same numerals. Additional or different features may be indicated by different numerals. In the second and third implementations of the main casing 63, the sleeve 44 is not shown; however, it is understood that the sleeve 44 is compatible with the second and third implementations and may function substantially similar to the sleeve 44 as described above with respect to the first implementation to create a smooth transition between the handpiece 24 and the battery and control module 21. When compared to the first implementation of the main casing 63 shown in FIG. 26, the second implementation of the main casing 63 as shown in FIG. 28, has a smaller distal barrel 60 and the third implementation as shown in FIG. 29 has a larger distal barrel 60. Additionally, the third implementation of the main casing 63, as shown in FIG. 28, has a plurality of projections that are rectangular shaped. Each of the plurality of projections includes an opening.

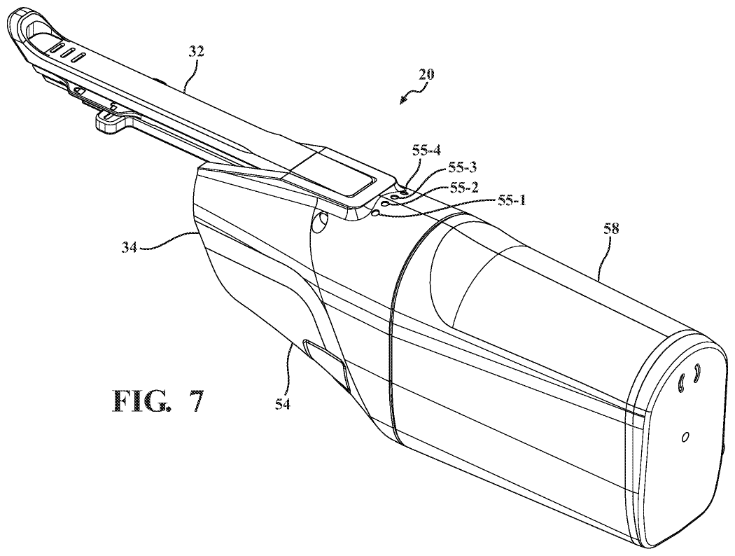

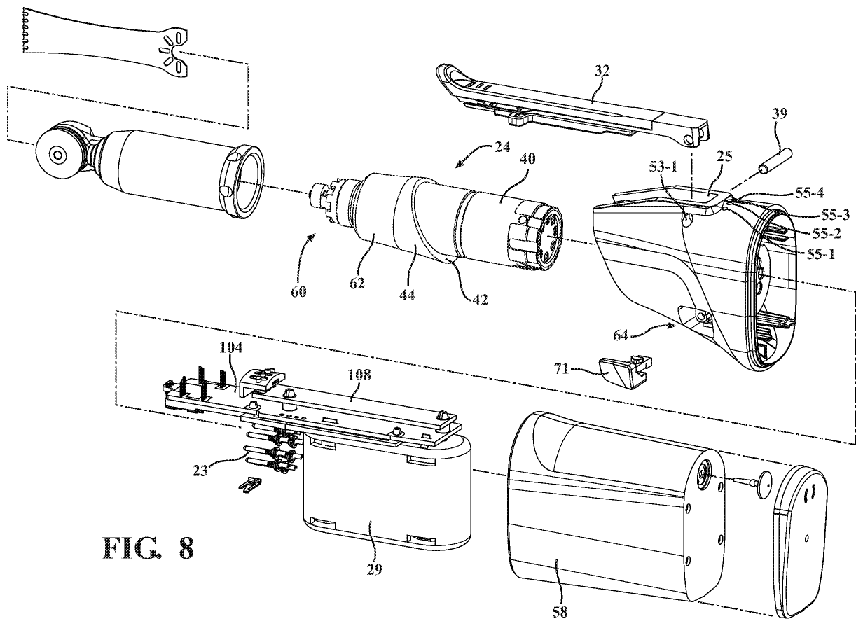

As discussed previously, the handswitch assembly 32 may be coupled to the upper aspect 48 of the device housing 22. In particular, the handswitch assembly 32 may be coupled to the flared portion of the device housing 22. With reference to FIG. 8, the flared portion of the device housing 22 may define a cavity 25. The flared portion 54 may be formed with a pair of apertures 53-1, and 53-2 (collectively, 53) adjacent to and on each side of the cavity 25. The handswitch assembly 32 may be partially seated within the cavity 25. The handswitch assembly 32 may be adjustable in length relative to the device housing 22.

With additional reference to FIGS. 9-12, the handswitch assembly 32 may include a mounting base 35, an elongate lever 15, a run-safe switch 37, and a lever extension 33. The mounting base 35 may be formed with a pair of openings 41 which facilitate the coupling of the handswitch assembly 32 to the device housing 22. A mounting member, for example a pin 39, may mount the handswitch assembly 32 to the flared portion 54. The pin 39 may be inserted through one of the pair of openings 41 of the flared portion 54 and through the pair of openings 41 of the mounting base 35 thereby coupling the handswitch assembly 32 to the device housing 22. The mounting base 35 defining a pivot axis to pivot about. An elongate lever 15 extends from the mounting base 35. A lever extension 33 is moveably coupled to the elongate lever 15 and adjustable relative to the elongate lever 15. In particular, the lever extension 33 may slide in the proximal and distal direction along the elongate lever 15.

A run-safe switch 37, which may be cross-shaped, may be slidably mounted to the elongate lever 15 and include a circular opening 43 which the magnet 45 is inserted through. The magnet 45 may be slidably mounted to the elongate lever 15 or mounted directly to the run-safe switch 37. The run-safe switch 37 may be engaged by the user as a safety switch to prevent inadvertent operation of the powered surgical tool 20 by moving the magnet 45 away from the handswitch sensor when the run-safe switch 37 is moved toward a distal end of the powered surgical tool 20. The lever extension 34 may be formed with a gripping portion 47 to assist the practitioner with gripping the powered surgical tool 20 and to prevent the practitioner's fingers from slipping off the lever extension 34. The gripping portion 47 may include an arcuate surface 49 and a plurality of ribs 51 disposed proximal to the arcuate surface 49. As shown, the plurality of ribs 51 includes three; however, any number or ribs are contemplated. The proximity of the run-safe switch 37 the lever extension 33, in particular, the gripping portion 47, provides the user with the advantage of being able to slide the run-safe switch 37 from or to safety mode without having to move their hand, or needing to use their other hand to place the powered surgical tool 20 in safety mode. The handswitch assembly 32 and battery and control module 21 may include features discussed in U.S. Pub. No. 2022/0175353, entitled, “Handswitch And Connector For Powered Surgical Handpiece,” the contents which are hereby incorporated by reference in their entirety.

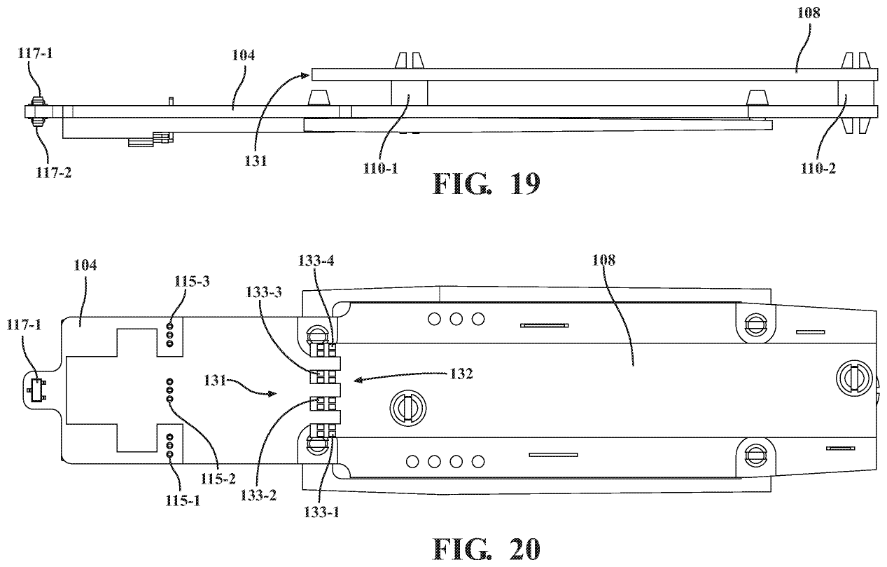

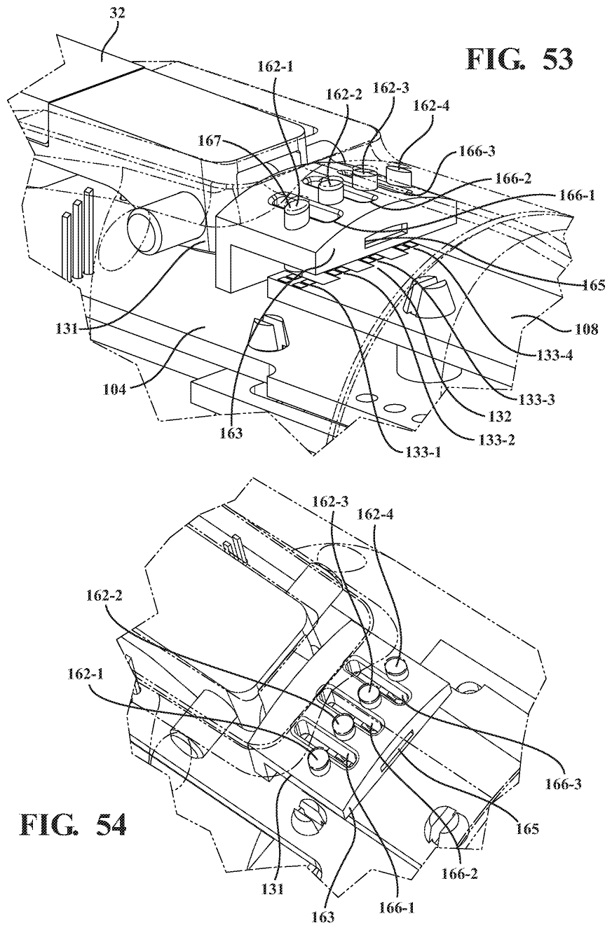

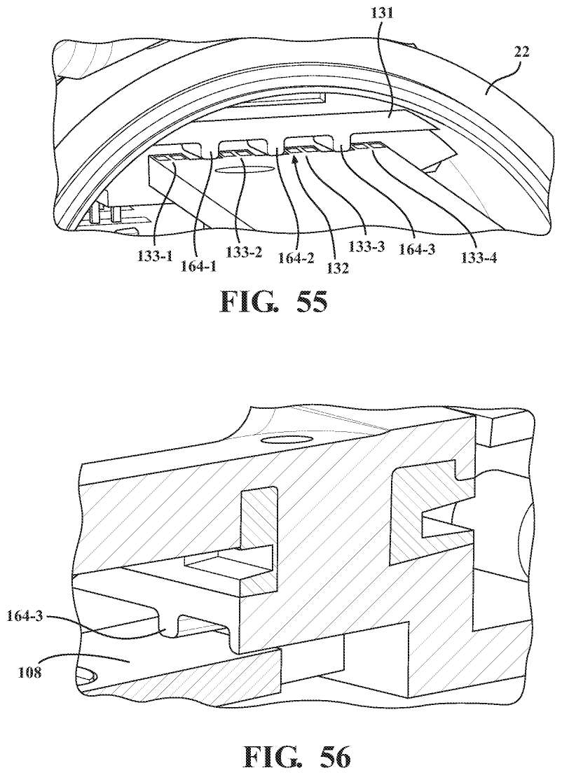

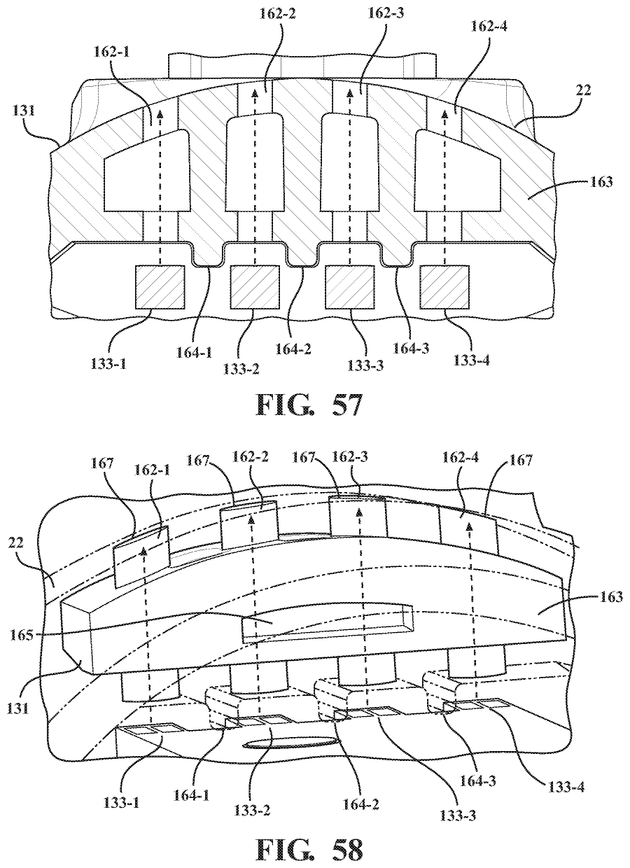

With reference to FIG. 7, 8, the flared portion 54 or other portion of the housing may be formed to include a first light aperture 55-1, a second light aperture 55-2, a third light aperture 55-3, and a fourth light aperture 55-4 (collectively, a plurality of light apertures 55) being adjacent to the cavity 25 where the handswitch assembly 32 is coupled to. The plurality of light apertures 55 are configured to allow for light generated from a plurality of light emitting diodes (LEDS) to be provided to the user, as will be discussed in greater detail with respect to FIGS. 19, 20, and 53-58.

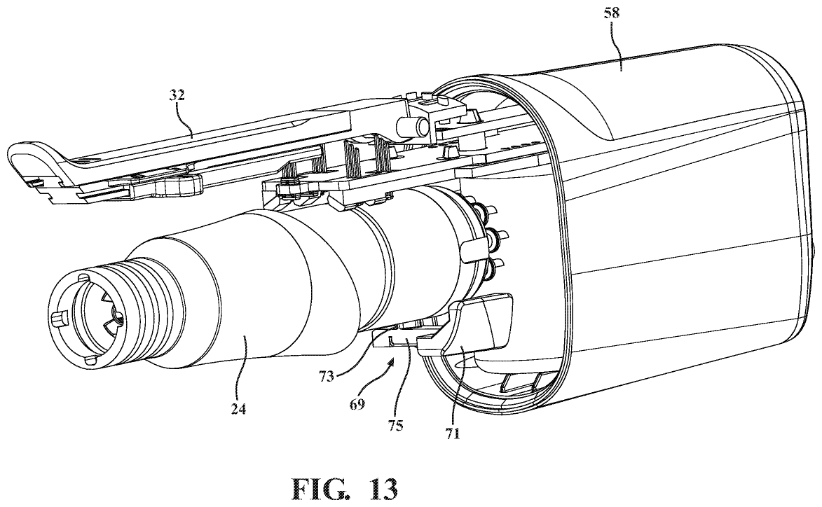

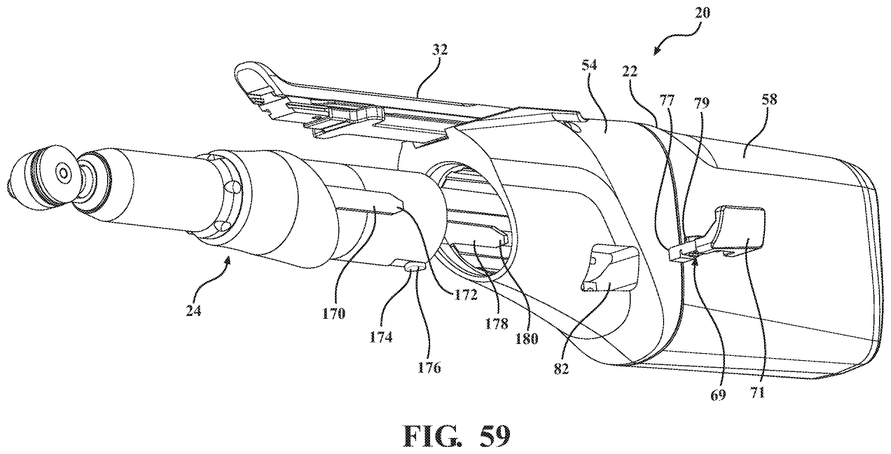

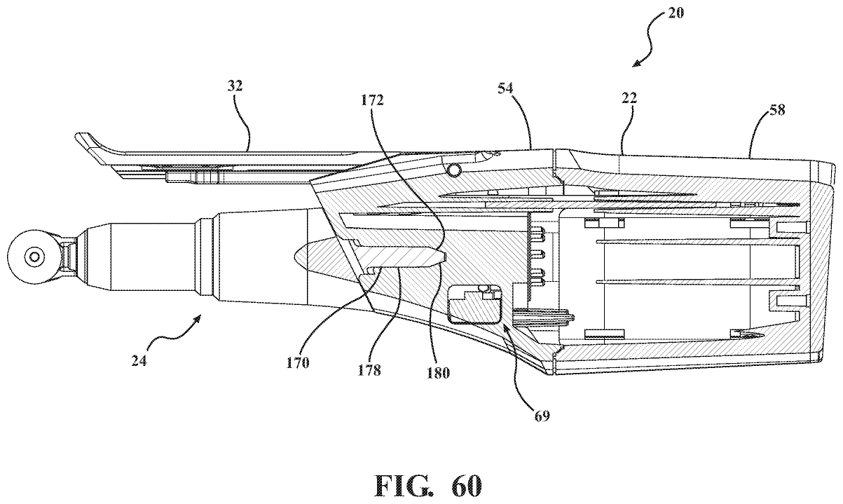

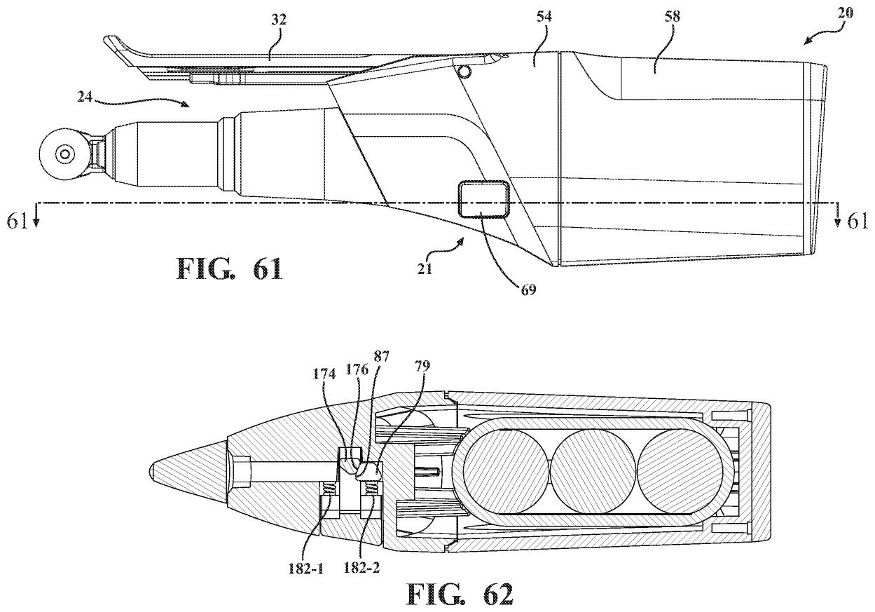

With reference to FIGS. 13-15 and 59-62, a latch assembly 69 cooperates with complementary features integral with handpiece 24 and the flared portion 54 to releasably hold the handpiece 24 in the recess 34. When the latch assembly 69 is in an engaged state, vibration and noise is reduced from the interaction between the handpiece 24 and the battery and control module 21. The flared portion 54 may include a first opening 81 and a second opening 82. The latch assembly 69 includes a first portion 71, a lock member 79, a pair of biasing members 182, an elongate portion 75, and a second portion 77. The first portion 71 is dimensioned to seat in and move in void space defined by the first opening 81 of the flared portion 54. The second portion 77 is dimensioned to seat in and move in void space defined by the second opening 82 of the flared portion 54. The first portion 71 and the second portion 77 may be connected by the elongate portion 75. The first portion 71 and the second portion 77 has a larger surface area than the second portion 77. The lock member 79 may define a tapered surface 87.

The handpiece 24 may include a pair of latch protrusions 170 that each include a tapered surface 172 and a notch 174 that includes a tapered surface 176. The notch 174 may be located closer to a proximal end of the handpiece 24 than the pair of latch protrusions 170. The pair of latch protrusions 170 may each have a distal end and a proximal end, with each of the proximal ends being arrow shaped. The pair of latch protrusions 170 may be radially offset from each other by at least 120 degrees.

An inner surface of the device housing 22 (surrounding the recess 34) may define a pair of channels 178, with each of the pair of channels 178 defining a tapered surface 180 that is complementary in shape to the tapered surfaces 180 of the pair of latch protrusions 170 of the handpiece. When the handpiece is fully seated within the recess 34 and the latch assembly 69 is in an engaged state, the lock member 79 is biased by the biasing members 182 such that the tapered surface 87 of the lock member 79 is actively urged against the notch 174 and the tapered surfaces 172 of the handpiece 24 are actively urged against the tapered surfaces 180 of the channel 178 of the device housing 22 such that vibration and noise caused from the interaction between the handpiece 24 and the battery and control module 21 is reduced. While the example is provided that the device housing 22 includes a pair of channels 178 and the handpiece includes a pair of latch protrusions 170, one of the pair of channels 178 and one of the pair of protrusions 170 may be omitted and a single channel 178 and a single latch protrusion 170 may still reduce vibration and noise caused from the interaction between the handpiece 24 and the battery and control module 21.

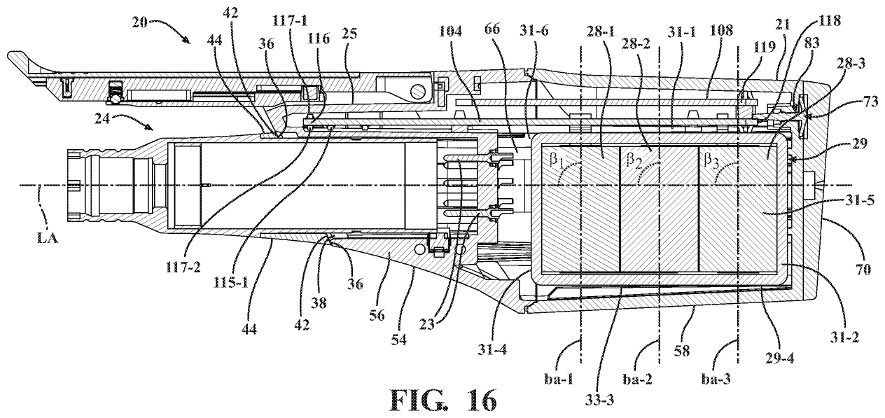

With reference back to FIG. 16, the base portion 58 is formed to include an opening 83 for venting of the void space. For example, the void space may be vented during sterilization. A cap 70 is coupled to the base portion 58 to cover the opening 83. A pressure relief valve 73 is at least partially inserted into the opening for facilitating venting of the void space during sterilization. The cap 70 covers the pressure relief valve 73 and may protect the pressure relief valve 73, e.g. from being damaged/hit, by jets from washer during washing. The pressure relief valve 73 may be configured to open at a predetermined pressure. When the valve opens, the is clearance between the pressure relief valve 73 and the cap 70. The cap 70 may include one or more cuts adjacent or proximal to the position of the pressure relief valve 73. The cuts allow the gas/air to be released.

The device housing 22 defines an interior 66 within which the battery cells 28 are disposed. Subsequent disclosure will be described as plural battery cells with three battery cells including a first battery 28-1 with a first longitudinal axis ba-1, a second battery 28-2 with a second longitudinal axis ba-2, and a third battery 28-3 with a third longitudinal axis ba-3 shown in certain figures; however, it is contemplated that more or less battery cells may be provided. The powered surgical tool 20 of the present implementation through advantageous arrangements of the battery cells 28 of the battery and control module 21 may provide and increased power to weight ratio, may move the center of mass 64 closer to the web of the hand of the user, may provide for improved balance when compared to similar systems, and overall improved ergonomics.

The motor controller 30 may be coupled to a first printed circuit board 104 or a second printed circuit board 108 that are disposed within the interior 66. The flared portion 54 may define the recess 34 that itself defines a longitudinal axis (LA) of the powered surgical tool 20. The base portion 58 may define the interior 66 in which the first battery 28-1, a second battery 28-2, and a third battery 28-3 are arranged in a manner such that a first longitudinal axis ba-1 of the first battery 28-1, the second longitudinal axis ba-2 of the second battery 28-2, and the third longitudinal axis ba-3 of the third battery 28-3 are substantially parallel with each other. Angles, β₁, β₂, and β₃ defined between the longitudinal axis (LA) of the powered surgical tool 20 and at least one each of the first longitudinal axis ba-1, the second longitudinal axis ba-2, and the third longitudinal axis ba-3 is 90 degrees or within a range of 90 degrees (e.g., 75 to 115 degrees). In other words, the longitudinal axis of one or more battery cells may be perpendicular to the longitudinal axis of the surgical tool (as defined by the recess of the housing). This layout of the battery cells relative to the axis of the surgical tool has unexpectedly been found to provide optimal balance configurations, sufficient power, and result in a compact design.

The battery cells 28 may also be positioned proximal to the saddle region 52 at a location to minimize moment forces about the hand. The battery cells 28 may be surrounded by an insulation layer 27. The insulation layer 27 may help to protect the battery cells 28 from excessive heat, for example, heat generated during autoclave sterilization, and also mechanical shocks and vibrations. The battery cells 28 and surrounding layer of insulation layer 27 may form a battery pack 29 (i.e., the battery cells 28 wrapped in insulation) that has a generally rectangular cross-sectional shape in the y-x plane. The battery pack 29 may form an outer periphery that has a shape that includes a first face 31-1, a second face 31-2, a third face 31-3, a fourth face 31-4, a fifth face 31-5, and a sixth face 31-6 (collectively, a plurality of faces 31). The faces 31 may include a curved surface where the faces join other faces so that the faces appear to merge into each other.

As mentioned, the battery cells 28 may be appreciably heavy owing to the power required, and therefore positioning the center of mass 64 of the battery cells 28 below the longitudinal axis facilitates the center of mass 64 of the powered surgical tool 20 being closer to the hand of the user, and preferably, as close as possible to the hand of the user.

The battery cells 28 may be short and thick cylindrical battery cells so as to allow for a more compact arrangement of the battery cells 28 that minimizes the footprint (e.g., in volume) of the control module. For example, each of the battery cells 28 may have a width close to 18 millimeters or within a range of 18 millimeters (e.g., 14 to 22 millimeters), or a diameter of less than 20 mm. Each of the cells may exhibit a height ranging between 26 and 34 mm. In some implementations, the cells exhibit a height of less than 32 mm. Each of the first battery, the second battery, and the third battery, and optionally, the fourth battery, may output a nominal cell voltage of at least 3.5 V or within a range of 3.6 V (e.g., 3.2 v to 4.0 v). Each cell may be capable of at least 11 or 13 A current draw.

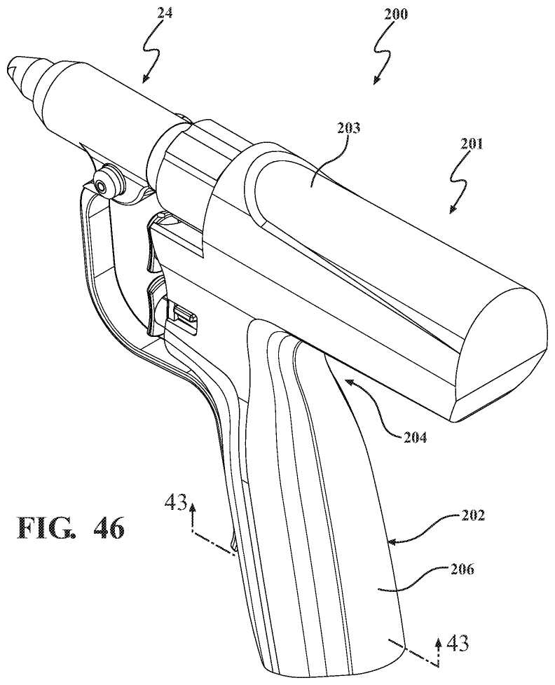

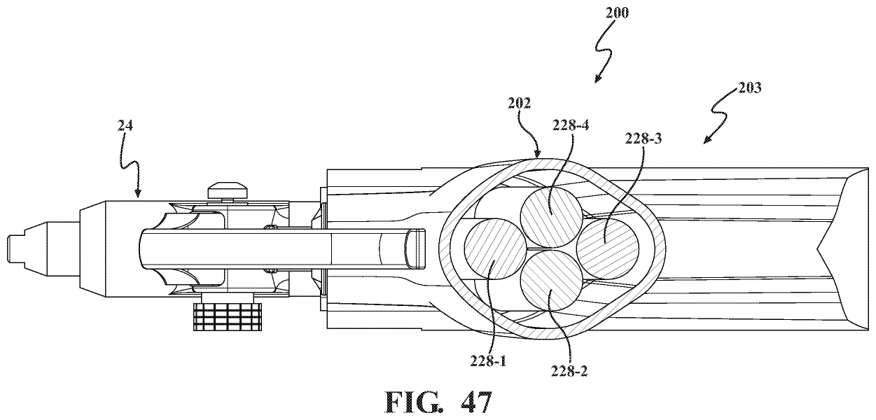

In one implementation, with reference to a pistol grip implementation of FIG. 47, the control module may include exactly four battery cells. In such an implementation, the compact battery powered surgical tool may exhibit a mechanical output power of at least 110 W. This control module may also have a weight of less than 280 g. This control module may exhibit a volume of less than 350 or less than 325 cm³. This volume measurement omits the handpiece and any tool coupled thereto. The four battery cells are collectively capable of producing an electrical output power of at least 160, 180, or 200 W. The four cells may be arranged in two columns, with two cells having their longitudinal axis aligned with one another in a first of the two columns and two cells having their longitudinal axis aligned with one another in a second of the two columns.

In another implementation, with reference to pencil grip implementation of FIG. 62, the control module may include exactly three cells. In such an implementation, the compact battery powered surgical tool exhibits a mechanical output power of at least 90 W, despite only having three cells. This control module may have a weight of less than 240 g. This control module may exhibit a volume of less than 185 or less than 175 cm³. This volume measurement includes the handswitch, but omits the handpiece and any tool coupled thereto. The three battery cells are collectively capable of producing an electrical output power of at least 120, 130, 140, or 150 W. The three cells may be optionally aligned such that the three cells are arranged such that a longitudinal axis of each cell is aligned with one another and the ends of the three cells are aligned with one another.

Surprisingly, the inventors of the present application realized that the selection of cell chemistry having a relatively lower onset of thermal runaway rating ranging could be used in the battery and control module despite the fact that the battery and control module must be able to safely withstand the temperatures of sterilization after performance of the surgical procedure. By selecting a cell having such a rating, the inventors were able to vastly improve the power output of the device, while simultaneously reducing the volume of the control module. In one exemplary configuration, the battery cells may include a cathode combination that includes nickel-manganese-cobalt. For example, the cathode combination may include one third nickel, one third cobalt, and one third manganese.

The onset of thermal runaway temperature is a temperature at which a chemical reaction starts inside the battery. If the battery reaches a temperature greater than its onset of thermal runaway temperature, the chemical reaction starts and the reaction generates additional heat. It is call runaway as there is no way to stop it. Removing any external heat source from the cell will not stop it, as the chemistry inside the cell fuels the reaction, and the cell can burn from the inside out.

Autoclaves can run at temperatures up to 139 degrees Celsius, which leaves only a 20 degree buffer. However, autoclaves and other sterilizers are not the only heat source. As cells degrade over their life, they generate higher internal resistance. If you combine the heat of the sterilizer, such as an autoclave with a battery having higher than normal internal resistance, there is a risk that the cell chemistry can exceed the onset of thermal runaway temperature, leading to a fire in a hospital.

Conventional battery modules may include Lithium-Iron-Phosphate cells, which have an onset of thermal runaway rating of at 220 degrees Celsius. This provides an extensive margin versus conventional autoclave temperatures.

The cells for the control modules described herein may be selected such that the onset of thermal runaway rating ranges from 140 to 180 degrees Celsius or from 140 to 160 degrees Celsius. In another configuration, the cells may be selected such that the cells have an onset of thermal runaway temperature rating that is less than 160 degrees Celsius. In another implementation, the cells for the control modules may have an onset of thermal runaway temperature rating ranging between 150 and 200 degrees C. or 160 degrees and 200 degrees C.

The battery and control modules are configured for low-temperature sterilizers, or at temperatures less than 80 degrees Celsius. The battery and control modules may not be compatible with autoclaveable sterilizers. The inventors realized that utilizing the battery cells with the recited onset of thermal runaway temperature ratings reduces sterilization options, but vastly improves power and ergonomics of the control modules.

As best illustrated by FIG. 22, the base portion 58 may include a first rib 129-1, a second rib 129-2, a third rib 129-3, a fourth rib 129-4, a fifth rib 129-5, and a sixth rib 129-6 (collectively, referred to as a plurality of ribs 129) disposed along the interior of the surface being shaped and dimensioned to assist in holding the battery pack 29 into place once placed in the interior 66 of the base portion 58. The battery pack 29 may abut each one of the plurality of ribs 129 once placed into the interior 66 of the base portion 58. The ribs 129 may also provide structural stiffness to the design.

With additional reference to FIGS. 17-23, the first printed circuit board 104 may be longer and wider than the second printed circuit board 108 and thus, has a larger surface area than the second printed circuit board 108. For example, the surface area of the first printed circuit board 104 may be at least 30, 40, or 50% more than the second printed circuit board 108. However, other suitable dimensions are contemplated. The first printed circuit board 104 and the second printed circuit board 108 are arranged in a stacked configuration which helps to minimize the footprint of the powered surgical tool 20, in particular, a small footprint of the battery and control module. The minimized footprint of the battery cells 28, including relatively short height of the battery cells 28, within the void space allows for the stacked configuration of the first and second printed circuit boards 104, 108 to fit within the void space above the battery cells 28.

The first printed circuit board 104 and the second printed circuit board 108 may be mechanically coupled to each other by a first fixation element 110-1 and a second fixation element 110-2 (collectively, fixation elements 110) to form the stacked configuration. In the exemplary configuration, the fixation elements are implemented as retention posts. The first printed circuit board 104 and the second printed circuit board 108 may be electrically connected in any suitable manner, such as with a PCB header. The first printed circuit board 104 may have a first longitudinal axis and the second printed circuit board may have a second longitudinal axis, with the first and second longitudinal axes being in alignment. The first and second printed circuit board 104, 108 may each have rigid back layers and are thus rigid printed circuit boards. However, other configurations, such as a flex printed circuit boards and rigid-flex printed circuit boards are contemplated for the first printed circuit board 104 and the second printed circuit board 108.

In the stacked configuration, a first surface 105 of the first printed circuit board 104 faces a first surface 107 of the second printed circuit board 108 while a second surface 109 of the first printed circuit board 104 faces the recess 34 of the flared portion 54 and a second surface 111 of the second printed circuit board 108 faces the handswitch assembly 32. The first printed circuit board 104 and the second printed circuit board 108 may be spaced apart by a margin selected so that the components coupled to the first surfaces 105 of the first printed circuit board 104 and the first surface of the second printed circuit board 108 do not interfere or bump into each other. In an example, this margin may be set from 3 millimeters (mm) to 16 mm or another suitable distance. Additionally, the first and second printed circuit boards 104, 108 may be electrically connected to each other. A proximal end 118 of the first printed circuit board 104 may be in line with a proximal end 119 of the second printed circuit board 108. Since the first printed circuit board 104 is longer than the second printed circuit board 108, a distal end 121 of the first printed circuit board 104 extends forward a proximal end 122 of the second printed circuit board 108. Furthermore, the second printed circuit board 108 may be positioned such that it does not extend beyond the first printed circuit board 104 in any direction beyond being spaced apart from the first circuit board as described above.

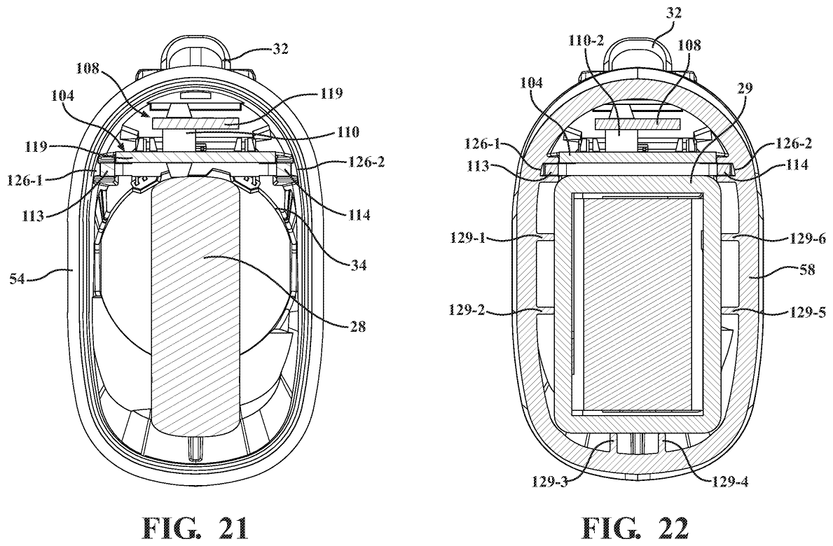

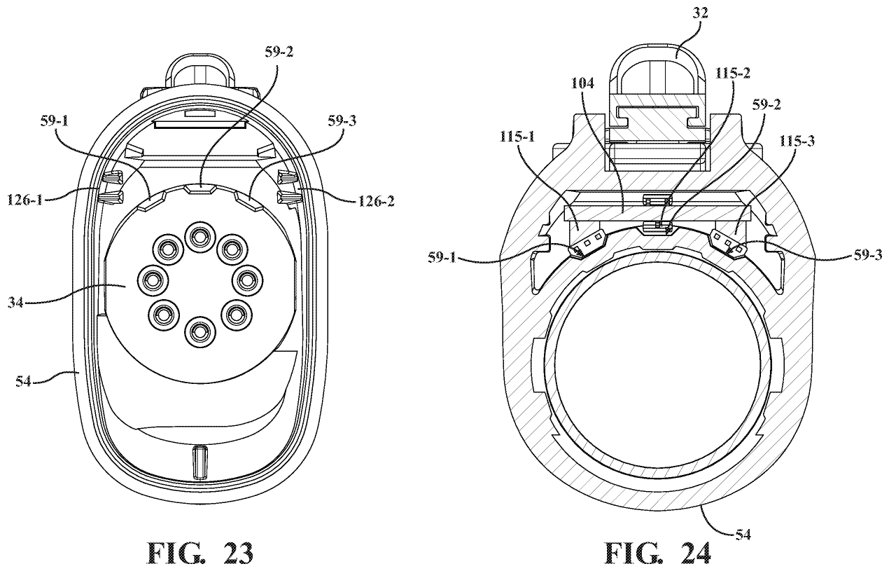

With particular reference to FIG. 17, a chassis 112 including a first portion 113 and a second portion 114 may be coupled to the first printed circuit board 104, in particular the second surface 109, to facilitate positioning and coupling of the first and second printed circuit boards 104, 108 to the device housing 22. As best illustrated at FIGS. 21 and 23, an internal surface of the flared portion 54 may be formed with a first pair of slots 126-1, 126-2 (collectively, a first pair of slots 126) adjacent to the recess 34. The first pair of slots 126 are located between the cavity 25 in which the handswitch assembly 32 is coupled to and the recess 34 in which the handpiece 24 gets inserted into. An internal surface of the base portion 58 may be formed with a second pair of slots 127. When the base portion 58 is coupled to the flared portion 54, the first pair of slots 126 is in alignment with the second pair of slots 127.

The first and second pair of slots 126, 127 facilitate the positioning of the first and second printed circuit boards 104, 108 by receiving the first and second portions 113, 114 of the chassis 112. In particular, the first portion 113 of chassis 112 is slid into one of the first and second pair of slots 126, 127 and the second portion 114 of the chassis 112 is slid into the other of the first and second pair of slots 126, 127. The distal end 121 of the first printed circuit board 104 may include a tip 116 which allows for the first printed circuit board 104 to be positioned closer to a tapered end 61 of the flared portion 54. The chassis 112 and the first and second pair of slots 126, 127 are sized and dimensioned to assist in locating the various electrical components of the first and second printed circuit boards 104, 108. Once the first and second portions 113, 114 of the chassis 112 have been slid into the first pair of slots 126 and the second pair of slots 127, the electrical components attached to the first and second printed circuit boards 104, 108 are in the desired locations, thus the powered surgical tool 20 of the present disclosure is easier to assembly when compared to systems of the prior art in which one or more of the electrical components may have to be individually placed. The first and second printed circuit boards 104, 108 may be located between a first face 31-1 of the plurality of faces 31 of the battery pack 29 and the device housing 22. In particular, the first printed circuit board 104 may be located closer to the handpiece 24 and the battery pack 29 than the second printed circuit board 108. While the example is provided that the flared portion 54 and the base portion 58 are formed with slots 126, 127, other alignment features are contemplated that would facilitate the placement and guidance of the first and second printed circuit boards 104, 108.

The motor sensors 115 may be coupled to the second surface 109 of the first printed circuit board 104 so that the motor sensors 115 face the motor when the handpiece 24 is inserted into the recess 34. As best illustrated by FIGS. 23 and 24, the internal surface of the recess 34 may be formed with a first recessed portion 59-1, a second recessed portion 59-2, and a third recessed portion 59-3 (collectively, a plurality of recessed portions 59). The recessed portions 59 are shown spaced approximately 30 degrees apart but may be spaced apart by any suitable difference. The motor sensors 115 are configured to measure a condition associated with an operating state of motor of the handpiece 24. For example, the motor sensors 115 may generate output signals representative of the rotational position of the rotor of the motor. One such sensor capable of generating signals representative of this rotor rotational position is a Hall effect sensor. A Hall effect sensor generates signals that vary with the sensed magnetic field. The magnetic field adjacent the rotor of the motor is a function of the rotational position of the rotor. As shown, the motor sensors 115-1, 115-2, and 115-3 are disposed at various varying positions nearing the distal end 121 of the first printed circuit board 104 to allow for optimal sensing of motor parameters of the handpiece 24. Each of these sensors is a Hall effect sensor that outputs an analog signal of the magnetic field sensed by the sensor. While three motor sensors are shown, any number of motor sensors may be used. The motor sensors 115 may include other sensors that may generate sensor signals as a function of the operating rate of the motor; the temperature of a component of the motor; the voltage applied across or the current applied to the motor. The motor sensors 115 may also measure a specific spectrum of light that the powered surgical tool emits as a function of the operating state of the motor.

One of the handswitch sensors 117, in particular a first handswitch sensor 117-1, may be coupled to the tip 116 of the first printed circuit board 104 at the first surface 105 while a second handswitch sensor 117-2 may be coupled to the tip 116 of the first printed circuit board 104 at the second surface 109. The handswitch sensors 117 measures the relative position of the magnet 45 of the handswitch assembly 32 by measuring magnetic field strength or direction between the handswitch sensors 117 and the magnet as the switch is depressed. It should be appreciated that the material forming the device housing 22 is material through which the magnetic fields are able to flow with attenuation and distortion levels that do not affect the ability of the handswitch sensor 117 to output signals representative of switch position. While several examples of mounting positions for the first handswitch sensor 117-1 and the second handswitch sensor 117-2 have been discussed, the first handswitch sensor 117-1 and the second handswitch sensor 117-2 may be mounted in another suitable manner.

With particular reference back to FIGS. 19, 20, and 53-58, a light guide 131 may be coupled to the second printed circuit board 108. It is certainly contemplated that the light guide could be coupled to any suitable printed circuit board with any control module or with a battery housing separate from a control module. The light guide 131 may allow light from a light emitting diode (LED) module 132 to be directed to the light apertures 55. The LED module 132 includes a plurality of light emitting diodes 133 coupled to the second printed circuit board 108 which are arranged so that light emitted from the light emitting diodes 133 is directed through the light apertures 55. In particular, the light guide 131 assists to ensure light emitted from one of the plurality of light emitting diodes 133 does not interfere with light emitted from an adjacent one of the plurality of light emitting diode 133. The light guide 131 includes a first protrusion 162-1, a second protrusion 162-2, a third protrusion 162-3, and a fourth protrusion 162-4 (collectively, a plurality of protrusions 162) which get inserted through the first light aperture 55-1, the second light aperture 55-2, the third light aperture 55-3, and the fourth light aperture 55-4. A surface of each of the plurality of protrusions 162 defines an outmost surface of the battery and control module 21. The first LED 133-1 is aligned with the first protrusion 162-1, the second LED 133-2 is aligned with the second protrusion 162-2, the third LED 133-3 is aligned with the third protrusion 162-3, and the fourth LED 133-4 is aligned with the fourth protrusion 162-4 so the light emitted from each of the respective LEDs 133 is directed to a respective protrusion 162.

The light guide 131 defines a first aperture 166-1, a second aperture 166-2, and a third aperture 166-3 (collectively, a plurality of apertures 166) between the plurality of protrusions 162. A filler polymer which is opaque may be disposed in the plurality of apertures 166 during manufacturing. The light guide 131 may also include a first rib 164-1, a second rib 164-2, and a third rib 164-3 (collectively, a plurality of ribs 164) that contact the second printed circuit board 108 near where the plurality of LEDs are mounted and separate each of the plurality of LED from one another. For instance, the first rib 164, prevents light emitted from the first LED 133-1 from interfering with light emitted from the second LED 133-2 and vice versa, the second rib 164-2 prevents light emitted from the second LED 133-2 from interfering with light emitted from the third LED 133-3 and vice versa, and the third rib 164-3 prevents light emitted from the third LED 133-3 from interfering with light emitted from the fourth LED 133-4 and vice versa. The filler polymer may also be disposed between each of the plurality of ribs 164.

A method of forming a light guide in a housing of a shell component of a powered surgical tool is also contemplated. The shell component may be any suitable hermetically-sealed housing configured for sterilization. The method may include providing a plurality of light emitting diodes on a circuit board mounted in a first portion of a shell component. While LEDs are contemplated here, other light sources that are mounted to the circuit board are also contemplated. The method may also include providing a light guide including a plurality of protrusions and defining an aperture between the plurality of protrusions, the light guide being transparent.

The method may include positioning the light guide relative to the circuit board such that the protrusions of the light guide are aligned with the LEDs and such that a portion of the light guide, such as ribs of the light guide, contacts the circuit board. The light guide may be further positioned relative to a mold of an injection molding tool such that the position of the light guide is fixed relative to the mold fixture. This prevents the light guide from moving during the injection molding process. More particularly, the mold include a shape that is complementary to a light guide coupler 165 (shown in FIG. 65) defined by the light guide such that the mold can fix the position of the light guide. The light guide coupler 165 is shown as an a channel, but the light guide coupler could also be a protrusion.

The method may further include applying a molten filler material to a cavity of the mold filler such that the molten filler contacts the light guide. The molten filler material fills the one or more apertures 166-1, 166-2, 166-3 and contacts the surfaces of the protrusions 162-1, 162-2, 162-3, 162-4. The light guide is essentially held in place while the opaque molten plastic flows all around it to fill all the gaps around the light guide until the housing is fully formed.

The molten filler may be opaque such that the light emitted from the LEDs is not able to pass through the filler material when it has been solidified. The molten filler material is applied such that at least one surface of each of the plurality of protrusions is not contacted by the molten filler material. With reference to FIG. 58, the plurality of protrusions include a surface that faces away from the circuit board, which might be defined as the emission surface 167. The filler material does not contact the emission surface 167. The emission surface 167 may define a portion of the outermost surface of the hermetically-sealed housing of the control module after the molten filler material has solidified. While the protrusions are shown as cylindrically-shaped protrusions, other shaped protrusions are also contemplated. The light guide may be injection molded separately from a transparent material.

The light guide and method of forming the light guide provide advantageous results. For example, by providing a single light guide that includes multiple light paths for multiple LEDs, the manufacturing is simplified, as there is only a need to position a single component, the single light guide, as opposed to separate light guides associated with each LED. Furthermore, by utilizing the filler material to contact the light guide, a hermetic seal can be formed between the light guide and the housing of the control module such that liquids cannot contact the circuit boards of the control module or device during sterilization or during use of the surgical tool.

In the illustrated embodiment, the plurality of LEDs 133 of the LED module 132 are shown to include a first LED 133-1, a second LED 133-2, a third LED 133-3, and a fourth LED 133-4; however, the LED module 132 may include more or less LEDs 133. The controller 30 may be configured to operate the LED module 132 to provide various indications regarding the operating state of the powered surgical tool 20 to the medical professional using the powered surgical tool 20 or a separate controller may be configured to control the LED module, such as an LED driver positioned on a board separate from the controller 30. The motor controller 30 may be configured to operate the LED module 132 to provide various indications regarding the operating state of the powered surgical tool 20 to the medical professional using the powered surgical tool 20.

The first LED 133-1 of the LED module 132 may be operable in a first state and a second state, with the first state and the second state being visually discernable. In the first state, the first LED 133-1 may be operated by the motor controller 30 to emit a first type of light, such as blue or green light, to indicate to the user that no errors have been detected by the motor controller 30. In the second state, the first LED 133-1 may be operated by the motor controller 30 to emit a second type of light, such as yellow light, to indicate to the user that an error has been detected. The second LED 133-2, the third LED 133-3, and the fourth LED 133-4 collectively may be configured to provide a state of charge (SOC) of the battery pack 29. Each of the second LED 133-2, the third LED 133-3, and the fourth LED 133-4 may be operable in a first state and a second state by the motor controller 30. Additionally, the fourth LED 133-4 may be operable in a third state.

In each of the first states, the motor controller 30 may be configured to control the second LED 133-2, the third LED 133-3, and the fourth LED 133-4, to emit light, such as green light. In each of the second states, the motor controller 30 may control the second LED 133-2, the third LED 133-3, and the fourth LED 133-4 so that no light is emitted. In the third state for the fourth LED 133-4, the motor controller 30 may control the fourth LED 133-4 to emit a different color light (e.g., red light) than the fourth LED 133-4 emits in the first state. When the second LED 133-2, the third LED 133-3, and the fourth LED 133-4 are in the first state, the battery pack 29 is considered to be fully charged (100% SOC). When only the third LED 133-3 and the fourth LED 133-4 are in the first state and the second LED 133-2 is in the second state, this may indicate to the medical professional that the SOC of the battery pack 29 is less than 100 percent but greater than ⅔ of 100% SOC.

When only the fourth LED 133-4 is in the first state and the second LED 133-2 and the third LED 133-3 are in the second state, this may indicate to the medical professional that the SOC of the battery pack 29 is less than ⅔ but greater than ⅓ of 100% SOC. When the fourth LED 133-4 is in the third state and the second LED and the third LED 133-3 are in the second state, this may indicate to the medical professional that the SOC of the battery pack 29 is less than ⅓ of 100% SOC. When all of the LED 133-2, 133-3, 133-4 are in the second state, this may indicate to the medical professional that the battery pack 29 does not contain sufficient SOC to operate the powered surgical tool 20 properly.

Although an exemplary configuration is described above with respect to the LED module 132 and interaction between the motor controller 30, other configurations are possible. For instance, all four of the LEDs 133 may be used to provide a status as to the SOC of the battery pack 29 and one of more of the LEDs may be operable in more than the states described above (e.g., the LEDs may be controlled to emit a variety of colors of light or emit light in a variety of different ways to provide a variety of different indications to the medical professional).

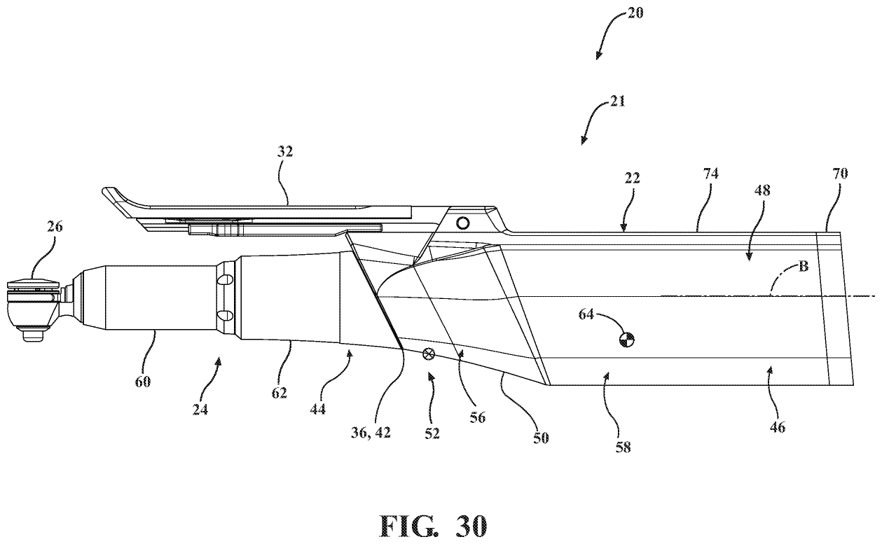

Referring now to FIGS. 30-32, a second implementation of the powered surgical tool 20 is illustrated. With the various implementation of the powered surgical tool 20 like features are indicated by the same numerals. Additional or different features may be indicated by different numerals. The surgical tool 20 includes the arcuate surface 50 being contoured to an adjacent portion of the handpiece 24 to define the saddle region 52. More particularly, the device housing 22 may include a flared portion 54 extending from the front surface 36, and a base portion 58 extending from the flared portion 54. The base portion 58 may be positioned proximal to the handswitch assembly 32. The flared portion 54 tapers in a distal direction or flares outwardly in a proximal direction, and the base portion 58 may have an axial section that is at least substantially constant. The flared portion 56 may be contoured to the sleeve 44 of the handpiece 24 with the handpiece 24 removably coupled to the device housing 22. As best shown in FIG. 30, the sleeve 44 of the handpiece 24 includes an outward taper in the proximal direction such that an interface or transition between the sleeve 44 and the flared portion 54 appears and feels smooth and uninterrupted. In such an arrangement, the smooth transition of the saddle region 52 advantageously accommodates hands of different sizes as well as different options for the user to comfortably position the hand along the powered surgical tool 20. For example, the user may prefer to pinch a distal barrel 60 of the handpiece 24 and support the sleeve 44 of the handpiece 24 with the web of the hand. With the web of the hand supporting the powered surgical tool 20 at support point (X), for example, a center of mass 64 of the powered surgical tool 20 is moved closer to the support point (X). Instances where the center of mass 64 is presently identified in the figures are to be understood as approximate locations, and further understood to constitute approximate locations of the center of mass with 64 or without the handpiece 24 removably coupled with the battery and control module 103.

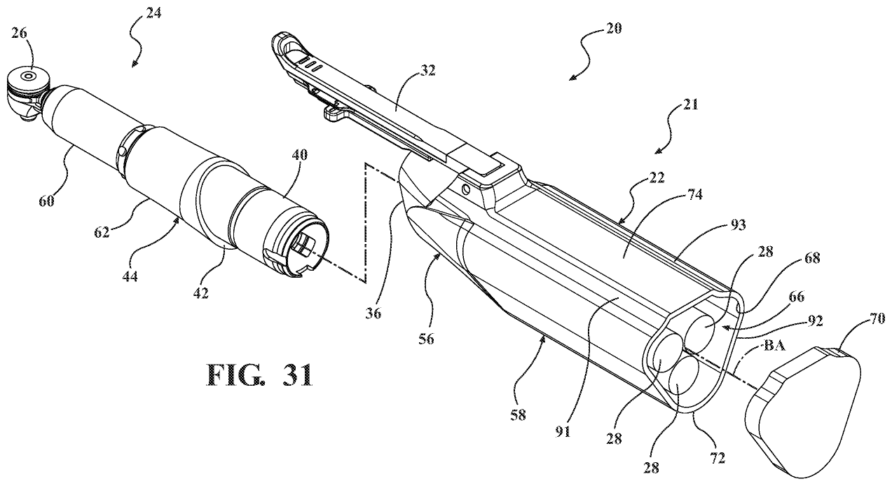

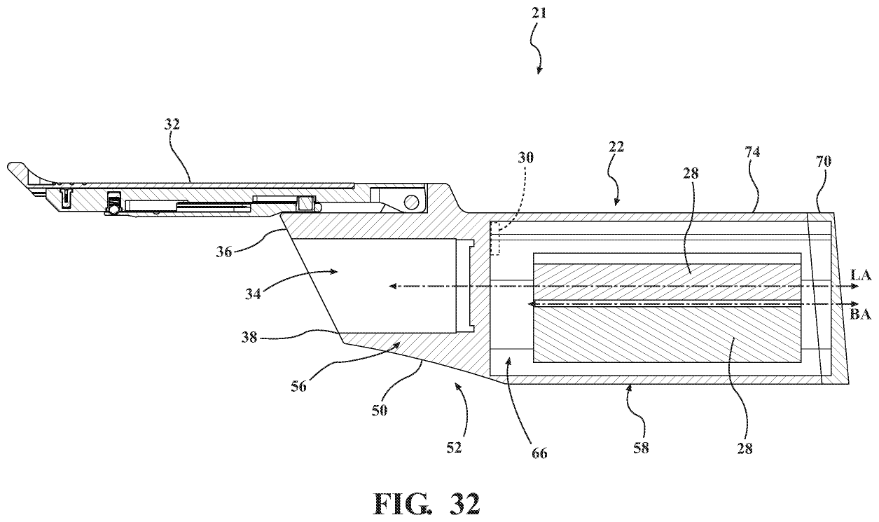

The powered surgical tool 20 of the present implementation further provides for lowering the center of mass 64 through advantageous arrangements of the battery 28 (or battery cells) of the battery and control module 21. Subsequent disclosure will be described as plural battery cells with three battery cells shown in certain figures; however, it is contemplated that more or less battery cells may be provided. With continued reference to FIGS. 31 and 32, the device housing 22 defines an interior 66 within which the battery cells 28 are disposed. The device housing 22 may define an opening 68 that opens distally into the interior 66. A cap 70 may be coupled to the device housing 22 to cover the opening 68 and seal the interior 66 from the ambient environment. The motor controller 30 may also be disposed within the interior 66 as well as additional subcomponents of the battery and control module 21. The flared portion 56 may define the recess 34 that itself defines a longitudinal axis (LA) of the powered surgical tool 20. The base portion 58 may define the interior 66 such that the interior 66 is offset downwardly relative to the recess 34. As a result, with the battery cells 28 oriented longitudinally within the interior 66, the battery cells 28 are arranged on a battery unit axis (BA) that is offset relative to the longitudinal axis to be closer to the hand of the user. The position of the desired center of mass is also influenced by the angle at which the device is held during surgery. The battery cells 28 may also be positioned proximal to the saddle region 52 at a location to minimize moment forces about the hand. As mentioned, the battery cells 28 may be appreciably heavy owing to the power required, and therefore positioning the battery cells 28—or at least their center of mass-below the longitudinal axis facilities the center of mass 64 of the powered surgical tool 20 being closer to the hand of the user, and preferably, as close as possible to the hand of the user.

As best shown in FIG. 31, the battery cells 28 may be arranged to be at least substantially parallel and adjacent to one another to define an outer periphery that is triangular in shape and define the battery unit axis about its center. Particularly where the battery cells 28 are cylindrical, the compact arrangement of FIG. 31 minimizes a footprint (e.g., in width and height) and equalizes lateral weight distribution of the powered surgical tool 20. Further, with the outer periphery of the battery cells 28 being rectangular in shape, several additional advantages are realized. First, the base portion 58 may be rectangular in shape complementary to the outer periphery of the battery cells 28. The rectangular shape of the base portion 58 results in a lower edge 72 that is curved and laterally centered. Consequently, the curve may be configured to comfortably rest within the web of the hand, if necessary, and the lateral centering of the lower edge 72 provides symmetry for lateral weight distribution. Second, the triangular shape of the base portion 58 provides an upper surface 74 that is at least substantially flat. The upper surface 74 being substantially flat improves line of sight to the surgical site and facilitates ergonomic handling in a hammer grip configuration to be described. Third, the base portion 58 being triangular in shape further includes side surfaces 91, 92 that meet the upper surface 74 at upper edges 93 that are contoured for comfort while also providing for proper indexing of the powered surgical tool 20 within the hand of the user.

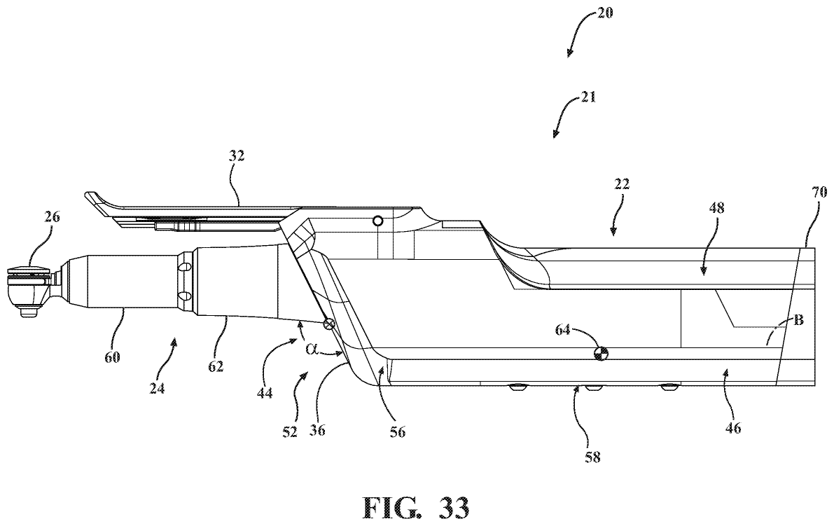

Referring now to FIGS. 33-35, a third implementation of the powered surgical tool 20 is shown with like numerals indicating like features. The powered surgical tool 20 includes the front surface 36 defining the opening 38 that opens into the recess 34 configured to removably receive the handpiece 24. The front surface 36 slopes proximally to the lower aspect 46 of the device housing 22. With the handpiece 24 removably coupled to the battery and control module 21, the front surface 36 may cooperate with the sleeve 44 of the handpiece 24 to define the saddle region 52. The transition from the sleeve 44 to the device housing 22 may be pronounced, for example, at an angle, a, of less than 135 degrees. The angle may be greater than 90 degrees such that the saddle region 52 remains contoured to receive the web of the hand. With the more pronounced transition, the front surface 36 may facilitate supporting the powered surgical tool 20 at steeper angles of approach to the surgical site.

Further owing to the more pronounced transition, the web of the hand is more likely to support the powered surgical tool 20 at the support point (X) shown in FIG. 33. Together with the arrangement of the battery cells 28 to be described, the vertical distance between the support point (X) and the center of mass 64 of the powered surgical tool 20 is minimal. In fact, FIG. 33 shows the support point (X) being above the center of mass 64 of the powered surgical tool 20.

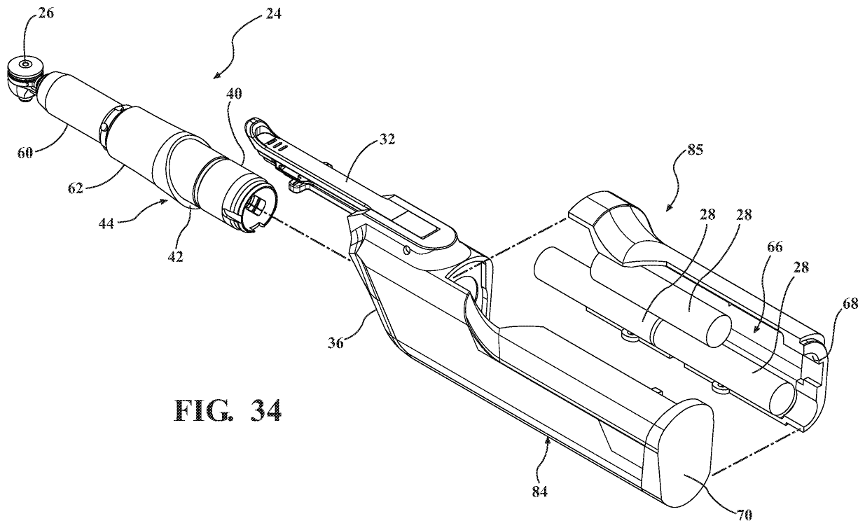

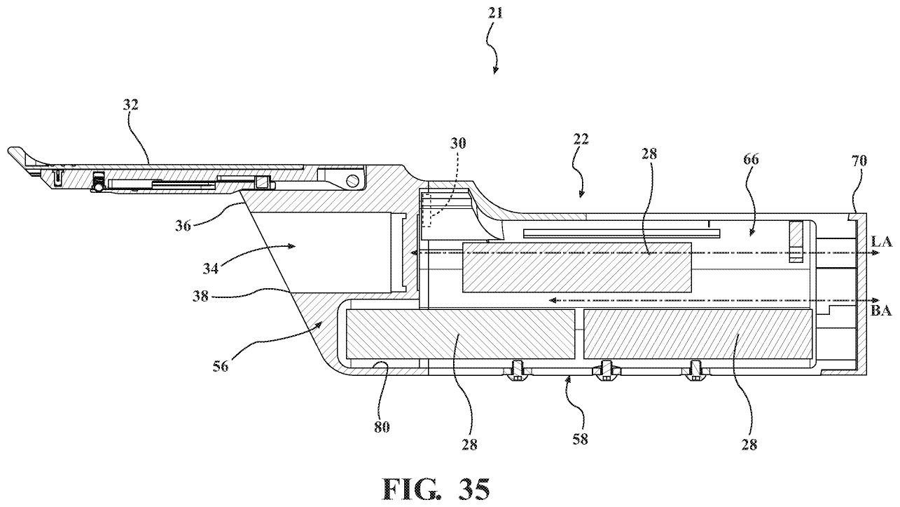

As best shown in FIGS. 33 and 34, the battery cells 28 may be in a stacked arrangement. More particularly, the device housing 22 includes opposing casings 84, 85 that define the interior 66 within which the battery cells 28 are disposed, and internal geometries of the device housing 22 provide for at least one of the battery cells 28 positioned atop another at least one of the battery cells 28. For example, one of the battery cells 28 may be positioned atop two of the battery cells 28, which further lowers the center of mass 64 of the powered surgical tool 20. While the FIGS. 33 and 34 illustrate opposing casings 84, 85 as coupled in a particular manner, it is understood that the device housing may be coupled together in a different manner. For example, instead of opposing casing 84, 85, the device housing could include a proximal portion and a distal portion that couple together to house the battery cells 28. The recess 34 defines the longitudinal axis (LA) of the powered surgical tool 20, and the battery cells 28 are arranged longitudinally on the battery unit axis (BA) that is offset from the longitudinal axis to be closer to the hand of the user. It is noted that a center of mass 64 of the battery cells 28 is below the battery unit axis shown in FIG. 35. To accommodate two of the battery cells 28 being positioned end to end without adding length to the device housing 22, the device housing 22 may define a counterbore 80 that opens into the interior 66. FIG. 35 shows the counterbore 80 sized to accommodate a portion of one of the battery cells 28. The stacked arrangement of FIGS. 33 and 34 provides for the device housing 22 being thinner in width while the lateral weight distribution of the battery cells 28 remains equal.

The thinner profile further facilitates improved line of sight as well as ergonomic handling the powered surgical tool 20 in the hammer grip configuration. The height being greater than the width may provide for proper indexing of the powered surgical tool 20 within the hand of the user. The upper surface 74 may be at least substantially flat to improve line of sight to the surgical site and facilitates ergonomic handling in the hammer grip configuration. The device housing 22 may include an upper contour 82 adjacent the upper surface 74 to further facilitate handling in the hammer grip configuration.

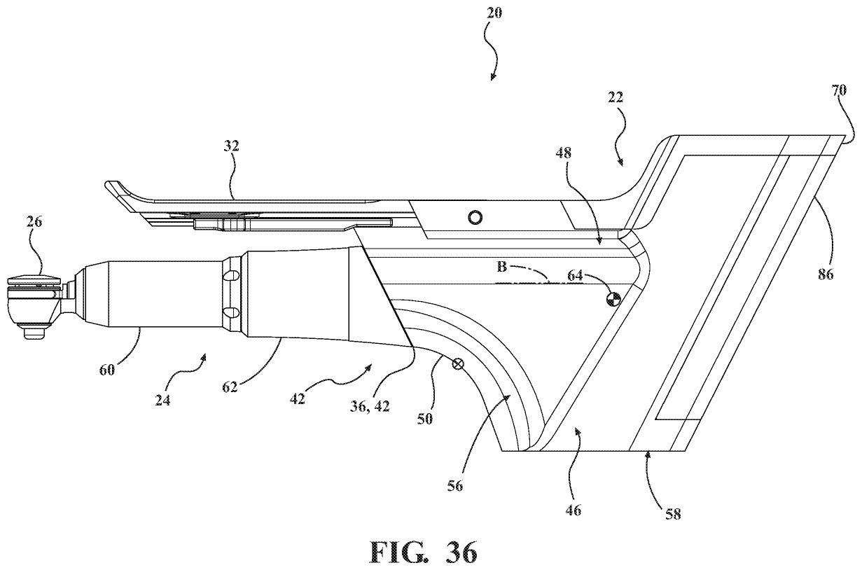

Referring now to FIGS. 36-38, the fourth implementation of the powered surgical tool 20 is shown with like numerals indicating like features. Among other advantageous features to be described, the center of mass 64 of the powered surgical tool 20 is positioned more distally to improve ergonomic handling of the powered surgical tool 20 in the pencil configuration. The device housing 22 includes the arcuate surface 50 that is contoured to an adjacent portion of the handpiece 24 to define the saddle region 52. The flared portion 54 extends from the front surface 36, and the base portion 58 extends in opposing directions from the flared portion 54. The flared portion 54 may be contoured to the sleeve 44 of the handpiece 24 with the handpiece 24 removably coupled to the battery and control module 21 such that the interface or transition between the sleeve 44 and the flared portion 54 appears and feels smooth and uninterrupted. The sleeve 44 and the flared portion 54 cooperate to define the saddle region 52. The saddle region 52 is sized to accommodate the web of the hand of the user with the handpiece 24 being configured to be supported by digits of the hand in the pencil grip configuration. Further, the saddle region 52 may be designed to cooperate with the upper surface 74 to enable the user to handle the powered surgical tool 20 in the hammer grip configuration.

The recess 34 of the device housing 22 defines the longitudinal axis (LA) of the powered surgical tool 20, and the interior 66 of the device housing 22 is shaped to house the battery cells 28 on the battery unit axis (BA) that is angled relative to the longitudinal axis. More particularly, the base portion 58 may include the lower aspect 46 and the upper aspect 48 that extend downwardly and upwardly from the flared portion 54, respectively. The device housing 22 may generally be considered T-shaped in form. The upper aspect 48 may be positioned above the handswitch assembly 32, and the cap 70 may be coupled to the upper aspect 48. A rear surface 86 may extend between the lower aspect 46 and the upper aspect 48 and define the tallest part of the device housing 22. The rear surface 86 may be substantially flat and parallel to the battery unit axis, or may include geometries for grasping and handling the powered surgical tool 20.

The angle, β₄, defined between the longitudinal axis and the battery unit axis may be less than 90 degrees, for example, within a range of 50 to 80 degrees. With the web of the hand supporting the powered surgical tool 20 at support point (X) within the saddle region 52, for example, a proximal-to-distal distance between the support point (X) and a center of mass 64 of the powered surgical tool 20 is lessened relative to other devices that are relatively more elongate in form. The longitudinally compact arrangement advantageously offers more balance in the hand of the user by reducing the moment forces about the hand, thereby lessening or eliminating a back-heavy feeling associated with known devices.

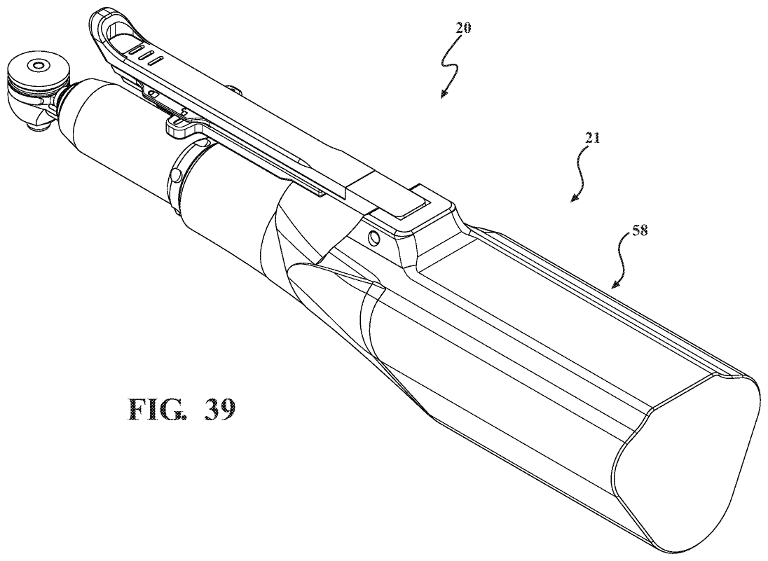

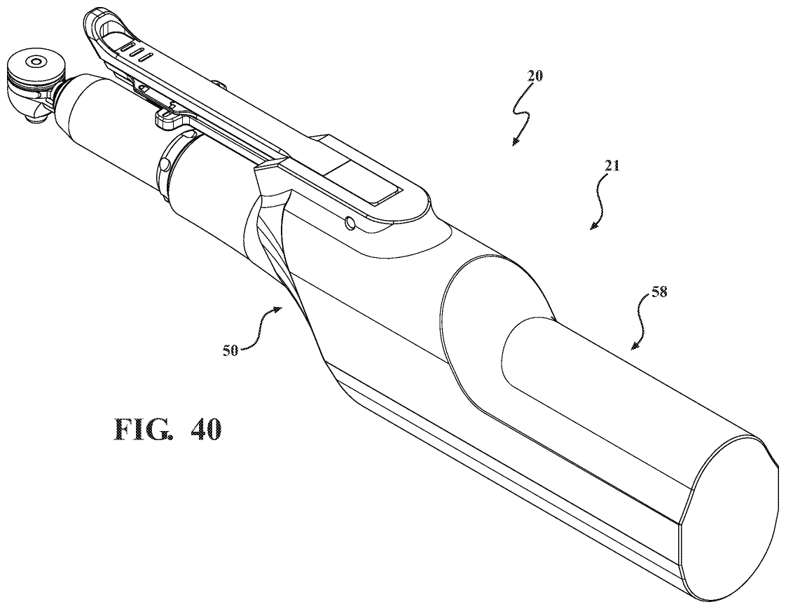

It should be appreciated that alternative dimensions and geometries of the device housing 22 are contemplated. For example, FIGS. 39-41 show implementations of the powered surgical tool 20 that respectively correspond generally to the three implementations previously described. The implementation of FIG. 39 includes the base portion 58 of the device housing 22 being shorter. The implementation of FIG. 40 includes the base portion 58 of the device housing 22 being shorter, and a differing contour to the flared portion 56. The shorter implementations of the base portion 58 provide for the center of mass 64 being more distal and closer to the hand of the user, thereby lessening or eliminating the back-heavy feeling. The implementation of FIG. 41 includes an upper surface of the cap 70 being coplanar with the upper aspect 48 of the device housing 22 that may provide for further improved line of sight.

FIG. 42-45 depict another implementation of the powered surgical tool 200 shown in a pistol configuration. A battery and control module 201 of the pistol configuration includes a barrel 203 and a handle 202. The handle 202 extends downwardly from the barrel 203. The handpiece may be inserted to a recess 216 of the barrel 203. Four battery cells, including a first battery 228-1, a second battery 228-2, a third battery 228-3, and a fourth battery 228-4 are disposed in the handle 202 which may be surrounded by an insulation layer 229 to form a battery pack 238. Battery and control module 231 has two switches 231, 233 that are spring-loaded. Both switches extend forward from the distally directed portion of the handle 202. The practitioner may actuate switches 231, 233 to control the operation of the tool unit. The second switch 233 is located below switch 231. In particular, one of the switches 231, 233 may cause the motor to run in reverse and the other of the switch 231, 233 may cause the motor to run in reverse. When both switches 231, 233 are actuated at the same time, the motor may oscillate. Although not illustrated, the battery and control module 201 may also include a toggle switch. The toggle switch may be configured as a safety switch which prevents switches 231, 233 from being actuated. The switches 231, 233 may include a magnet which gets moved when the switches 231, 233 actuated by the user as discussed in greater detail below. The battery and control module 201 may include a pressure relief valve 237 and a cap 239 that function substantially similar to the pressure relief valve 73 and cap 70 discussed with respect to the pencil configuration.

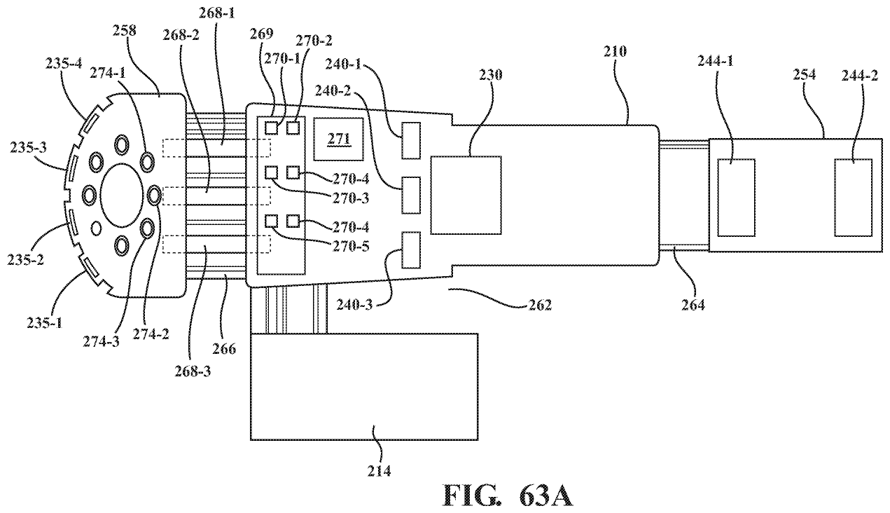

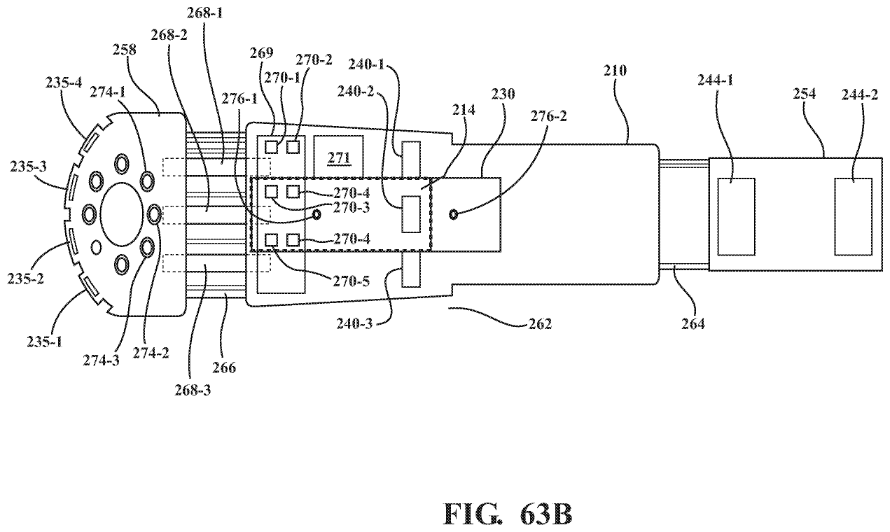

With reference to FIG. 63A, a first printed circuit board 210, a second printed circuit board 214, and a fourth printed circuit board 258 may be disposed in the barrel 203 while a third printed circuit board 254 is disposed in the handle portion. The printed circuit boards 210, 214, 254, and 258 may be connected to each other in any suitable manner. For example, the first printed circuit board 210, the second printed circuit board, 214, the third printed circuit board 254, and the fourth printed circuit board 258 may be provided as a single rigid flex PCB. As depicted, the first printed circuit board 210 is connected to the second printed board 214 by a first flexible portion 262, the first printed circuit board 210 is connected to the third printed circuit board 254 by a second flexible portion 264, and the first printed circuit board 210 is connected to the fourth printed circuit board 258 by at third flexible portion 266.

A first motor sensor 240-1, a second motor sensor 240-2, and a third motor sensor 240-3 (collectively, a plurality of motor sensors) 240 and the motor controller 230 may be coupled to the first printed circuit board 210. The motor sensors 240 may be Hall-effect sensors and may be similar to the previously described motor sensors 115. A switching module 269 including a first MOSFET 270-1, a second MOSFET 270-2, a third MOSFET 270-3, a fourth MOSFET 270-4, a fifth MOSFET 270-5, and a sixth MOSFET 270-6 (collectively, a plurality of MOSFETs 270) may also be coupled to the first printed circuit board. Although the disclosure contemplates MOSFETs as the switching components that are coupled to the first printed circuit board 210, other suitable transistors or switching components may be used. Additionally, other electrical components may also be coupled to the first printed circuit board 210 or any of the second printed circuit board 214, the third printed circuit board 254, and the fourth printed circuit board 258. The first printed circuit board 210 may include a first trace 268-1, a second trace 268-2, and a third trace 268-3 (collectively, a plurality of traces 268) that connect the plurality of MOSFETs 270 to motor terminals 274-1, 274-2, and 274-3 of the fourth printed circuit board 258. The plurality of MOSFETs 270 may be used in controlling the direction of operation of the motor of the handpiece 24, for example, in a forward direction or in a reverse direction. A plurality of pins 223 may also be disposed in the barrel 203 and may inserted through the plurality of terminals 274 to connect the battery and control module 201 to the handpiece 24.

The second printed circuit board 214 may include one or more electrical components for programming and debugging the motor controller 230. As such, the second printed circuit board 214 and the first flexible portion 262 may be removed prior to finishing assembly of the powered surgical tool 200. The second printed circuit board 214 may include one or more electrical components for programming and debugging the motor controller 230. As such, the second printed circuit board 214 and the first flexible portion 262 may be removed prior to finishing assembly of the powered surgical tool 200. The fourth printed circuit board 258 may include a first motor terminal 274-1, a second motor terminal 274-2, and a third motor terminal 274-3 (collectively, a plurality of motor terminals 274) which are connected via a first trace 268-1, a second trace 268-2, and a third trace 268-3 (collectively, a plurality of traces 268) to the plurality of MOSFETs 270. A first light emitting diode 235-1, a second light emitting diode 235-2, a third light emitting diode 235-3, and a fourth light emitting diode 235-4 (collectively, a plurality of light emitting diodes 235) may be coupled to the fourth printed circuit board 258. The plurality of LEDs 235 may be controlled by the motor controller 230 similar to the plurality of light emitting diodes 133 discussed with respect to the pencil configuration. The housing of the barrel 203 may be formed with a first aperture 246-1, a second aperture 246-2, a third aperture 246-3, and a fourth aperture 246-4 (collectively, a plurality of apertures 246). The fourth printed circuit board 258 is arranged so that the plurality of light emitting diodes 235 are aligned with the plurality of apertures 246. The arrangement of the plurality of LEDs 235 on the barrel 203 close to the cap 239 allows for optimal viewing when the medical professional is engaging the powered surgical tool 200 at the surgical site.

A first handswitch sensor 244-1 and a second handswitch sensor 244-2 (collectively, a plurality of handswitch sensors 244) may be disposed on the third printed circuit board 254. The handswitch sensors 244 may be Hall-effect sensors and may be similar to the previously described handswitch sensors 117. The third printed circuit board 254 is disposed inside the handle 202. In particular, the third printed circuit board 254 is disposed in close proximity to the switch 231, 233 so that the plurality of handswitch sensors 244 may sense a state of the switches 231, 233, such as when the switches 231, 233 have been actuated by the user.

With reference to FIG. 63B, an alternative implementation of the first printed circuit board 210, the second printed circuit board 214, the third printed circuit board 254, and the fourth printed circuit board 258 is shown. Like features are indicated by the same numerals. Additional or different features may be indicated by different numerals. In the alternative implementation of the first printed circuit board 210, the second printed circuit board 214, the third printed circuit board 254, and the fourth printed circuit board 258, the second printed circuit board 214, the first flexible portion 262 has been omitted and replaced a first fixation element 276-1 and a second fixation element 276-2 (collectively, fixation elements 276) to form the stacked configuration. The first printed circuit board 210 and the second printed circuit board 214 may be electrically connected by board-to-board headers.

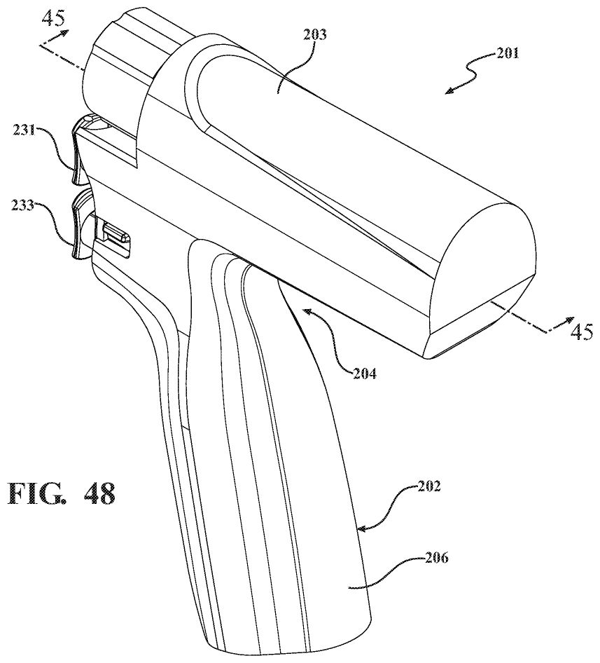

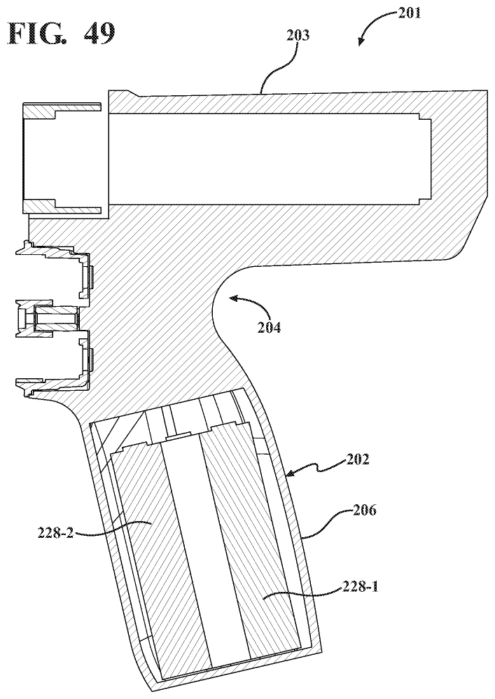

FIGS. 46 and 48 are perspective views of the powered surgical tool 200 in which the device housing is arranged in different pistol grip configurations than discussed with respect to FIGS. 42-45. With the various implementation of the powered surgical tool 200 like features are indicated by the same numerals. Additional or different features may be indicated by different numerals. A handle 202 of the battery and control module 201 may define a saddle region 204 that extends distally forward of a rear surface 206 of the handle 202. The rear surface 206 of the handle 202 may be angled relative to vertical with the powered surgical tool 200 in the operative position. The battery cells 228 may be disposed within the handle 202. There may be four battery cells 228 that are arranged adjacent one another to define an outer periphery that is a diamond in shape, as shown in FIG. 47. Alternatively, FIG. 49 reflects an arrangement where three of the battery cells 228 (two shown) are arranged adjacent one another to define the outer periphery that is substantially triangular in shape, and a fourth of the battery cells is stacked atop the three battery cells within the handle 202 of the battery and control module 201.

Referring to FIG. 16 again, the battery and control module 21 may include a plurality of conductive terminals 23, such as at least three or at least five conductive terminals. These conductive terminals 23 are used to provide power and communication between the controller and rechargeable battery module of the battery and control module 21 and the motor of the handpiece 24. The conductive terminals 23 need to be secured within the battery and control module 21 such that a portion of the conductive terminals 23 are outside of hermetic seal provided by the battery and control module 21 and a portion of the conductive terminals 23 are within the hermetic seal provided by the battery and control module 21. The conductive terminals 23 may be mounted to a circuit board, such as the first printed circuit board 104 or second printed circuit board 108 that is provided within housing that provides the hermetic seal.

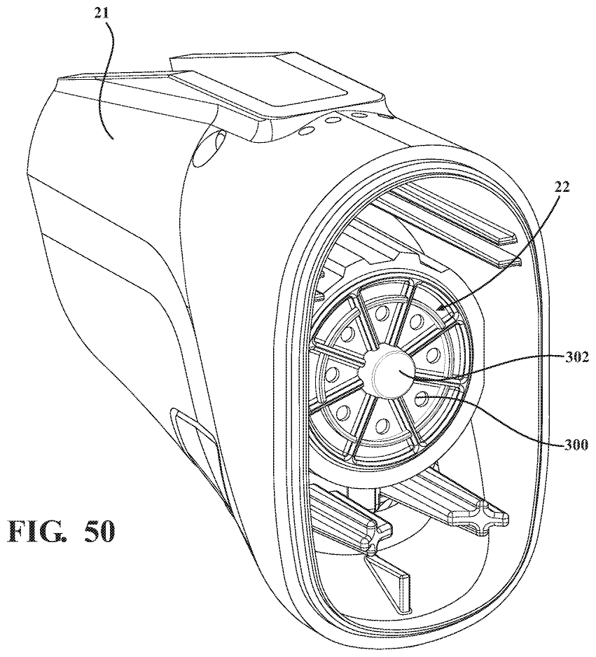

Referring now to FIG. 50, a rear view of a portion of the battery and control module 21 is provided. This rear view shows terminal apertures 300 surrounding a central region 302. In the view of FIG. 50, the conductive terminals 23 are hidden from view, but, if inserted, would be shown disposed in the terminal apertures 300.

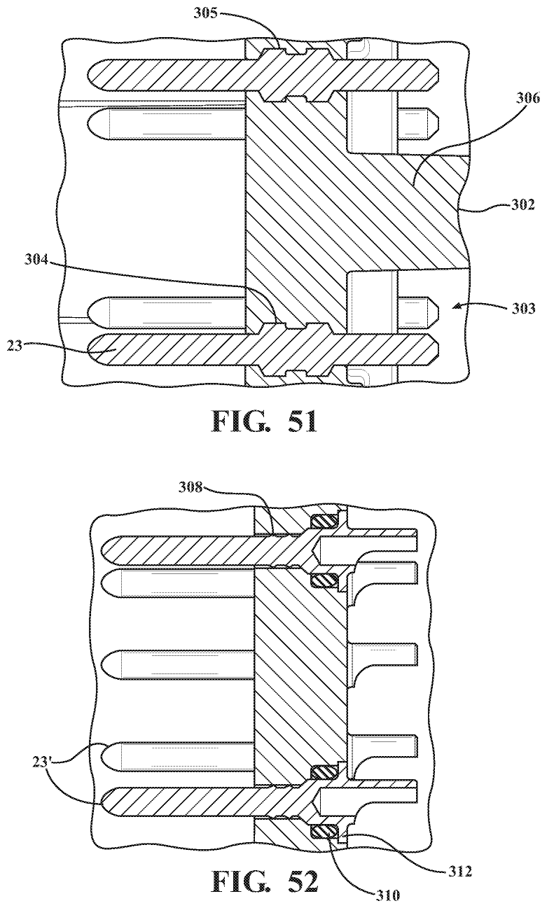

In this configuration, referring to FIG. 51, when formed, the control module 21 would include at least three conductive terminals 23 that extend through the device housing 22 for establishing a electrical connection between the circuit board and the motor of the handpiece. The plurality of conductive terminals 23 spaced apart from one another to define an array 303 of conductive terminals 23. The array 303 may surround the central region 302. The array 303 may be approximately circular in shape. Each of the conductive terminals 23 having a first end and a second end and a central region between the first end and the second end.

The control module is configured such that device housing 22 directly contacts at least three conductive terminals 23 to form a housing-terminal interface 304. The conductive terminals 23 may each include a collar 305 to facilitate the seal formed at the housing-terminal interface 304. The housing-terminal interface 304 defines a hermetic seal, such that liquid may not enter between the device housing 22 and the conductive terminals 23 and contact the electrical components within the device housing 22, such as the first printed circuit board 104 or the second printed circuit board 108. The housing-terminal interface 304 consist of a conductive material and the thermoplastic. In some constructions, the housing-terminal interface 304 may be free of an elastomeric sealing member, such as an o-ring. The control module 21 may exhibit higher-quality knit lines at the housing-terminal interface 304. The device housing 22 may include a mold exit artifact 306 disposed within the central region 302 defined by the array of conductive terminals In this instance, the device housing 22 is formed from a thermoplastic materials, such as a polyphenylsulfone thermoplastic.

As an alternative to the configuration shown in FIG. 51 in which the conductive terminals 23′ are hermetically sealed with housing via the molding process along, in FIG. 52, the conductive terminals 23′ are press fit into the device housing 22 after the housing 22 is formed. The conductive terminals 23′ may include an enlarged diameter portion 308 along with an elastomeric seal 310 positioned about each conductive terminal 23′.

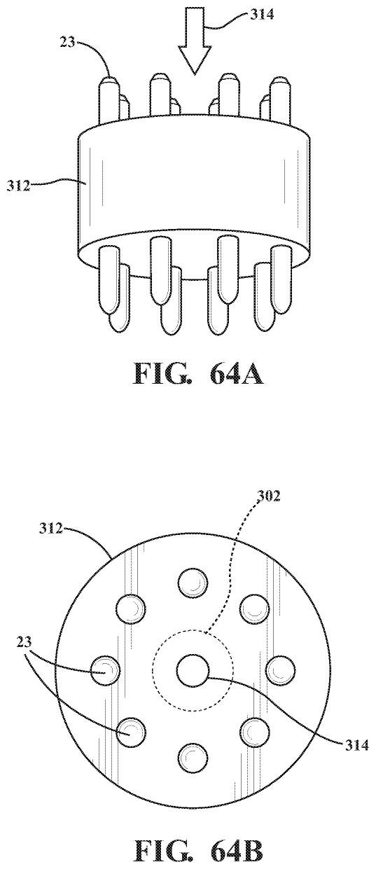

The present disclosure also relates to a method of forming a hermetic seal between the conductive terminals 23 and the device housing 22 such that the battery and control module 21 is configured for sterilization. Referring to FIGS. 64A and 64B, the method may include mounting the first end of the at least three conductive terminals in a mold fixture 312 such at the second end of the least three conductive terminals extend outward from the mold fixture 312, the at least three conductive terminals are positioned in the mold fixture to define the array 303 of conductive terminals surrounding a central region 302 between the at least three conductive terminals.

The method may further include injecting a molten filler material in the central region of the array of conductive terminals while the conductive terminals are mounted in the mold fixture 312 such that the molten filler material surrounds the median section of each of the at least three conductive terminals 23 and form a hermetic seal therewith while avoiding low quality knit lines on at least three conductive terminals 23. The molten filler material may be a polyphenylsulfone thermoplastic. In order to inject the molten filler material in the central region 302 of the array 303 of conductive terminals 23, the method may include positioning an injection molding gate 314 in the central region 302.

By injecting molten filler molten material in the central region 302 of the plurality of conductive terminals 23 in the mold fixture, the molten filler material can surround the median section of the conductive terminals before the molten filler material can cool significantly such that only high quality knit lines are formed at the interface. Poor quality knit lines, such as those that may be formed if the filler material was injected from a location other than the central region, can compromise the seal between the solidified form of the molten filler material and the plurality of conductive terminals and be susceptible to degradation with repeated chemical sterilization cycles.

Clause 1—A powered surgical tool comprising: a handpiece comprising a motor; a battery and control module comprising: a motor controller; a battery; a switch in communication with the motor controller and configured to receive an input from a user to cause power to be drawn from the three battery cells and supplied to the motor; and a device housing defining a cavity sized to removably receive the handpiece to establish electrical communication between the motor and the motor controller, an interior within which the battery is disposed, and a saddle region configured to support a web of a hand of the user, wherein the cavity defines a longitudinal axis of the device housing, wherein the battery is arranged about a battery unit axis that is offset from the longitudinal axis to be closer to the hand of the user.

Clause 2—The powered surgical tool of clause 1, wherein the battery is three battery cells arranged adjacent to one another to define an outer periphery that is substantially triangular in shape, and wherein the device housing comprises a base portion that is substantially triangular in shape to be complementary to the outer periphery of the three battery cells, and optionally, wherein the three battery cells are exactly three battery cells.

Clause 3—The powered surgical tool of clause 2, wherein the base portion comprises a lower edge of the substantially triangular shape that is curved and laterally centered with the powered surgical tool being supported in an operative position.

Clause 4—The powered surgical tool of any one of clauses 1-3, wherein an upper surface of the device housing is substantially flat and configured to be engaged by a thumb of the hand of the user in a hammer grip configuration.

Clause 5—The powered surgical tool of clause 4, wherein an adjacent side surface of the device housing is substantially flat and configured to be engaged by digits of the hand of the user in the hammer grip configuration, and wherein an edge between the upper surface and the adjacent side surface is rounded to accommodate the web of the hand of the user in the hammer grip configuration.

Clause 6—The powered surgical tool of clause 1, wherein the device housing comprises a base portion defining geometries within the interior that supports one of the battery cells above another one of the battery cells with being the battery cells being oriented substantially parallel to the longitudinal axis.

Clause 7—A powered surgical tool comprising: a handpiece comprising a motor; a battery and control module comprising: a motor controller; battery cells; a switch in communication with the motor controller, the switch being configured to receive an input from a user to cause power to be drawn from the battery cells and supplied to the motor; and a device housing coupled to the switch and defining a cavity sized to removably receive the handpiece to establish communication between the motor and the battery cells and control module, and further defining an interior within which the battery cells are disposed, the cavity defines a longitudinal axis of the device housing, wherein the device housing comprises geometries within the interior that supports one of the battery cells above another one of the battery cells with the battery cells being oriented substantially parallel to the longitudinal axis.

Clause 8—The powered surgical tool of clause 7, wherein the battery cells are three battery cells, and wherein a first and a second of the three battery cells are positioned end to end, and a third of the three battery cells is positioned above the first and second battery cells.

Clause 9—The powered surgical tool of clause 7 or 8, wherein the device housing further defines a counterbore in communication with the interior, and wherein a portion of one of the battery cells is disposed within the counterbore.

Clause 10—The powered surgical tool of clause 9, wherein the counterbore is positioned below the cavity.

Clause 11—The powered surgical tool of any one of clauses 7-10, wherein axes of the battery cells are coplanar with one another and the longitudinal axis.

Clause 12—The powered surgical tool of any one of clauses 7-11, wherein the device housing comprises a flared portion comprising an arcuate surface defining a saddle region configured to support a web of a hand of the user with the handpiece supporting digits of the hand of the user in a pencil grip configuration.

Clause 13—A powered surgical tool including comprising: a handpiece comprising a motor; a battery and control module comprising: a motor controller; a battery; a switch in communication with the motor controller, the switch being configured to receive an input from a user to cause power to be drawn from the battery and supplied to the motor; and a device housing coupled to the switch and comprising a flared portion defining a cavity defining a longitudinal axis and configured to removably receive the handpiece, and a base portion extending from the flared portion and defining an interior, wherein the flared portion further defines a saddle region sized to accommodate a web of a hand of a user in a pencil grip configuration, wherein the battery is elongated and disposed within the interior of the base portion on a battery unit axis that is angled relative to the longitudinal axis.

Clause 14—The powered surgical tool of clause 13, wherein an angle between the longitudinal axis and the battery unit axis is less than 90 degrees, and optionally, within a range of 50 to 80 degrees.

Clause 15—The powered surgical tool of clause 13 or 14, wherein the battery is three battery cells arranged to be substantially parallel and adjacent to one another to define an outer periphery that is triangular in shape.

Clause 16—The powered surgical tool any one of clauses 13-15, wherein the base portion comprises an upper aspect that is disposed above the flared portion.

Clause 17—The powered surgical tool of any one of clauses 13-15, further comprising a cap coupled to the base portion, wherein the cap is at least substantially coplanar to an upper surface of the switch.

Clause 18—A powered surgical tool comprising: a handpiece comprising a motor; a battery and control module comprising: a motor controller; a battery; a switch in communication with the motor controller, the switch being configured to receive an input from a user to cause power to be drawn from the battery and supplied to the motor; and a device housing coupled to the switch and defining a cavity sized to removably receive the handpiece to establish communication between the motor and the battery and control module, and further defining an interior within which the battery is disposed, wherein the device housing comprises a flared portion comprising an arcuate surface contoured to an adjacent portion of the handpiece so as to define a saddle region sized to accommodate a web of a hand of a user in a pencil grip configuration with the handpiece supporting digits of the hand of the user in the pencil grip configuration.

Clause 19—The powered surgical tool of clause 18, wherein the cavity defines a longitudinal axis of the device housing, and wherein the battery is positioned on a battery unit axis offset from longitudinal axis to be closer to the hand of the user.

Clause 20—The powered surgical tool of clause 18 or 19, wherein the battery comprises three battery cells arranged adjacent to one another to define an outer periphery that is substantially triangular in shape, and wherein the device housing comprises a base portion that is triangular in shape complementary to the outer periphery of the three battery cells.

Clause 21—The powered surgical tool of clause 18 or 19, wherein the device housing comprises geometries within the interior that supports the battery cells in a stacked arrangement in which one of the battery cells is above another one of the battery cells to be substantially parallel to the longitudinal axis.

Clause 22—The powered surgical tool of clause 18, wherein the cavity defines a longitudinal axis of the device housing, and wherein the device housing comprises a base portion defining the interior with the battery disposed within the interior on a battery unit axis that is angled relative to the longitudinal axis.

Clause 23—A device housing for a powered surgical tool as depicted and described herein.

Clause 24—A powered surgical tool comprising a device housing defining channels, a motor sensor disposed in the channels, and a housing insert configured to secure the motor sensor to the device housing as depicted and described herein.

Clause 25-Methods of assembling, supporting, and/or manipulating a powered surgical tool with a hand of a user as depicted and described herein.

Clause 26—A powered surgical tool including a plurality of LEDs mounted in a hermetically-sealed housing, the tool comprising: a housing defining a void space, the housing including a light guide, the light guide defines a plurality of protrusions, and the light guide defines an aperture between the protrusions, the light guide being transparent, a surface of the protrusions defining a portion of the outermost surface of the housing; a filler polymer disposed in the aperture, the filler plastic being opaque; a printed circuit board disposed in the void space; a plurality of light emitting diodes, the light emitting module being coupled to the printed circuit board, each of the plurality of light emitting diodes with a respective one of the plurality of protrusions so that light emitted from a light emitting diode does not interfere with light emitted from an adjacent light emitting diode. In such a configuration, the housing could be the battery and control module housing, or may be a battery housing only, or a tool housing only. This mounting can provide manufacturing advantages to any type of housing that includes a plurality of LEDs and that needs to be hermetically-sealed in a manner suitable for sterilization.

The foregoing description is not intended to be exhaustive or limit the invention to any particular form. The terminology which has been used is intended to be in the nature of words of description rather than of limitation. Many modifications and variations are possible in light of the above teachings and the invention may be practiced otherwise than as specifically described.

1. A powered surgical tool comprising:
a handpiece including a motor; and
a battery and control module including:
a module housing having a recess for removably receiving the handpiece, the module housing defining a void space;
a rechargeable battery module disposed in the void space;
a first printed circuit board disposed in the void space;
a second printed circuit board disposed in the void space, the second printed circuit board being coupled to the first printed circuit board, the second printed circuit board and the first printed circuit board being arranged in a stacked configuration, wherein the first printed circuit board and the second printed circuit board are both positioned on a same side of the rechargeable battery module; and
a controller configured to regulate power drawn from the rechargeable battery module based on user input, the controller being mounted to at least one of the first printed circuit board and the second printed circuit board.

2. The powered surgical tool of claim 1, wherein the battery and control module is constructed so as to isolate the void space from effects of a sterilization process.

3-14. (canceled)

15. The powered surgical tool of claim 1, further comprising a motor sensor being mounted to one of the first printed circuit board and the second printed circuit board and configured to output a motor sensor signal representative of a state of the motor.

16. The powered surgical tool of claim 15, wherein the controller is further configured to receive the motor sensor signal and control the motor based on the motor sensor signal.

17. The powered surgical tool of claim 16, wherein the motor sensor is a Hall effect sensor and the first printed circuit board includes a distal portion and a proximal portion, the motor sensor being coupled to the distal portion so that the motor sensor is positioned proximate to the motor in order to sense a motor parameter.

18. The powered surgical tool of claim 17, wherein the motor sensor is further defined as a first motor sensor and the motor sensor signal is further defined as a first motor sensor signal, the powered surgical tool further comprising a second motor sensor and a third motor sensor being coupled to the distal portion so that the second motor sensor and the third motor sensor are positioned proximate to the motor in order to sense the motor parameter.

19. The powered surgical tool of claim 18, wherein the first printed circuit board including a first surface and a second surface, wherein the motor sensor is mounted to the second surface of the first printed circuit board and faces the recess.

20. The powered surgical tool of claim 19, wherein the controller is mounted to the first printed circuit board.

21-23. (canceled)

24. The powered surgical tool of claim 1, wherein the first printed circuit board and the second printed circuit board are rigidly coupled to each other by one or more fixation elements.

25. The powered surgical tool of claim 1, wherein a surface area of the first printed circuit board is greater than a surface area of the second printed circuit board.

26. The powered surgical tool of claim 25, wherein both of the first printed circuit board and the second printed circuit board each have a rigid back layer.

27. The powered surgical tool of claim 26, wherein the first printed circuit board has a first longitudinal axis and the second printed circuit board has a second longitudinal axis, the first longitudinal axis being aligned with the second longitudinal axis.

28.-70. (canceled)

71. A powered surgical tool having a pistol grip, the powered surgical tool comprising:
a handpiece including a motor; and
a battery and control module including:
a module housing defining a barrel portion and a handle portion, the barrel portion having a recess for removably receiving the handpiece, the handle portion extending downwardly from the barrel portion, the module housing defining a void space;
a rechargeable battery module disposed in the void space;
a first printed circuit board disposed in the void space;
a second printed circuit board disposed in the void space, the second printed circuit board being coupled to the first printed circuit board, the second printed circuit board and the first printed circuit board being arranged in a stacked configuration, wherein the first printed circuit board and the second printed circuit board are both positioned on a same side of the rechargeable battery module; and
a Hall-effect sensor disposed in the void space and being mounted to one of the first and the second printed circuit board and configured to output a motor sensor signal representative of a state of the motor; and
a controller disposed in the void space, the controller being configured to regulate power drawn from the rechargeable battery module based on user input, the controller being mounted to at least one of the first printed circuit board and the second printed circuit board.

72. The powered surgical tool of claim 71, wherein the Hall-effect sensor is mounted to the second printed circuit board, the second printed circuit board being positioned farther from the handle portion of the module housing than the first printed circuit board.

73. The powered surgical tool of claim 72, wherein the first printed circuit board defines a longitudinal axis that is offset from parallel to an axis defined by the recess of the barrel portion.

74. The powered surgical tool of claims 72-73, further comprising a trigger assembly positioned adjacent the handle portion and including a movable trigger, the tool further comprising a third circuit board including a trigger sensor, the third circuit board positioned in the module such that movements of the movable trigger is detectable by the trigger sensor.

75. The powered surgical tool of claim 74, further comprising a fourth circuit board positioned proximal to the recess of the barrel portion, the fourth circuit board including terminals for connecting engaging a plurality of electrical receptacles on the handpiece and at least one LED.

76. The powered surgical tool of claim 75, wherein the second printed circuit board includes a plurality of transistors and motor traces, the motor traces coupled to the transistors.

77.-117. (canceled)

118. A powered surgical tool having a pistol grip, the powered surgical tool comprising:
a handpiece including a motor; and
a battery and control module including:
a module housing defining a barrel portion and a handle portion, the barrel portion having a recess for removably receiving the handpiece, the handle portion extending downwardly from the barrel portion, the module housing defining a void space;
a plurality of battery cells disposed in the void space;
a first rigid printed circuit board disposed in the void space;
a second rigid printed circuit board disposed in the void space, wherein the first printed circuit board has a first longitudinal axis and the second printed circuit board has a second longitudinal axis, the first longitudinal axis being aligned with the second longitudinal axis, and wherein the first and second printed circuit boards are arranged in a stacked configuration; and
a Hall-effect sensor disposed in the void space and being mounted to one of the first and the second printed circuit board and configured to output a motor sensor signal representative of a state of the motor; and
a controller disposed in the void space, the controller being configured to regulate power drawn from the plurality of battery cells based on user input, the controller being mounted to at least one of the first printed circuit board and the second printed circuit board.

119. The powered surgical tool of claim 118, wherein the first printed circuit board and the second printed circuit board are electrically connected.

120. The powered surgical tool of claim 119, wherein the first printed circuit board and the second printed circuit board are electrically connected with a pin header.
